(19) United States
(12) Patent Application Publication
(10) Pub. No.: US 2008/0171117 A1
(43) Pub. Date: Jul. 17, 2008

(54) METHODS FOR REDUCING MICROBIAL CONTAMINATION IN SEAFOOD PROCESSING

(75) Inventors: Stephen P. Mixon, Montgomery, TX (US); Dennis M. Smithyman, Shreveport, LA (US); Francis Dautreuil, Seabrook, TX (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Tasker Products Corp., Danbury, CT (US)

(21) Appl. No.: 11/874,034

(22) Filed: Oct. 17, 2007

Related U.S. Application Data
(63) Continuation-in-part of application No. 11/750,223, filed on May 17, 2007.
Application No. 11/750,223 is a continuation-in-part of application No. 11/674,588, filed on Feb. 13, 2007.
Application No. 11/674,588 is a continuation of application No. 10/922,604, filed on Aug. 20, 2004, now Pat. No. 7,192,618.
(60) Provisional application No. 60/801,494, filed on May 17, 2006.
(60) Provisional application No. 60/547,991, filed on Feb. 26, 2004.
(60) Provisional application No. 60/829,852, filed on Oct. 17, 2006.

(51) Int. Cl. A23B 4/12; C23G 1/02; A23B 4/08

(52) U.S. Cl. 426/332; 134/41; 99/517

(57) ABSTRACT

The present invention is generally directed to methods for reducing microbial population on food, especially seafood, during processing. Provided are methods of seafood processing that involve contacting seafood with a disinfection composition comprising an acid, a buffer, and an antimicrobial metal. Such methods can result in reduced pathogen load, reduced spoilage odor, and prolonged shelf-life of seafood. Also provided are systems for seafood processing employing said disinfectant compositions.

### CROSS-REFERENCE TO RELATED APPLICATIONS

This application claims priority from U.S. Provisional Application Ser. No. 60/829,852, filed on Oct. 17, 2006, which is incorporated herein by reference in its entirety.

### STATEMENT REGARDING FEDERALLY SPONSORED RESEARCH OR DEVELOPMENT

Not Applicable.

### FIELD

The present invention generally relates to reduction of pathogen load in seafood processing.

### INTRODUCTION

Pathogen contamination of seafood is a major concern of seafood processors in the U.S. and the rest of the world. Seafood is subject to rapid spoilage and is not safe to eat if not eaten within a short time of harvesting, i.e., within a couple of days, or treated to high energy processes such as canning or freezing which may change the texture and flavor of the seafood markedly. Seafood spoilage is mainly due to the rapid growth of bacteria in fresh seafood, particularly bacteria such as the psychrotrophic type. These bacteria grow rapidly, e.g., to a level of 106, within a few days. They also produce hydrogen sulfide, which adds a noxious odor to the seafood.

Seafood is processed primarily to convert the animal's muscles into meat, to remove the unwanted components of the seafood (blood, viscera, head, skin/shell), and to keep microbiological contamination at a minimum. The ultimate quality of the final product depends not only on the condition of seafood when arriving at the plant, but also on how the seafood is handled during processing. Various approaches have been utilized to lower pathogen prevalence on seafood. Some targeted areas for pathogen reduction include hold tanks, rinse systems, and immersion chillers.

Thus various researchers have attempted to find ways of preserving fresh seafood to prolong its shelf life to up to a week or more without causing deterioration in the texture or flavor, and while maintaining safe levels of bacteria. Chemical techniques for preserving seafood have been studied (see e.g., U.S. Pat. No. 5,196,221; U.S. Pat. No. 6,383,541; U.S. Pat. No. 5,389,390). Chemical preservation combined with packaging under a modified atmosphere has met with some success in decreasing the bacterial spoilage of fresh seafood.

Seafood processing, and disinfectants used therein, are discussed generally in Welt (1995) Seafood Regulations Compliance Manual, Springer, ISBN: 0412987511; Shahidi et al. (1997) Seafood Safety, Processing, and Biotechnology, CRC, ISBN: 1566765730; Haard and Simpson (2000) Seafood Enzymes: Utilization and Influence on Postharvest Seafood Quality, CRC, ISBN: 082470326X; Pigott (1990) Seafood, Food Science and Technology, CRC, ISBN: 0824779223; Bonnell (1994) Quality Assurance in Seafood Processing, Springer, ISBN: 0442008791; and Hall (1997) Fish Processing Technology, Springer ISBN: 0751402737.

Thus there exists a current need in the seafood processing industry for methods to reduce pathogen load in seafood processing and prolong shelf-life of seafood, without causing substantial deterioration in desirable organoleptic characteristics.

### SUMMARY

Accordingly, the present inventors have succeeded in discovering that an acidic buffered antimicrobial metal-containing disinfection composition decreases microbial contamination in seafood processing, decreases onset and progression of seafood odor, and extends the shelf-life of treated seafood. The present invention is generally directed to methods for reducing microbial population on food, especially seafood, during processing. The methods include the use of particular disinfection compositions suited for processing of food products, preferably meat processing, and more preferably seafood processing, at or during one or more processing steps. These disinfection compositions are generally non-oxidizing, acidic, buffered, copper-containing, food-safe disinfectants that can function efficiently in high temperature, high organic load, aqueous environments.

One aspect of the present invention is a method of reducing a microbial population on seafood during processing. Such method includes the step of contacting seafood during processing with a disinfection composition comprising an acid, a buffer, and an antimicrobial metal. The amount of disinfection composition used and the contact time is that sufficient to reduce a microbial population. The seafood can be, for example, submersed in or sprayed with the disinfection composition.

Various embodiments also provide for contacting the seafood with the disinfection composition during or immediately after one or more seafood processing steps. These steps can include harvesting, storing, holding, stunning, de-heading, eviscerating, skinning, chilling, trimming, washing, glazing (before the seafood is frozen, i.e., freeze glazing), packaging, transporting, and/or displaying.

Other embodiments further provide for recycling of seafood processing water containing the disinfection composition. These methods include the additional steps of recovering at least a portion of the disinfection composition contacted with the seafood; adding a sufficient amount of disinfection composition to yield a recycled disinfection composition; and contacting the seafood with the recycled disinfection composition during or immediately after processing steps listed above.

Some embodiments provide for contacting seafood with the disinfection composition (and/or recycled disinfection composition) at least during the steps of holding the seafood and chilling the seafood as well as immediately after trimming the seafood. For example, the seafood can be contacted with the disinfection composition or the recycled disinfection composition at least during holding, stunning, chilling, and glazing and immediately after trimming the seafood.

Another aspect of the invention provides for reducing a microbial population on seafood processing equipment. Such method includes the step of contacting a device used in seafood processing with a disinfection composition comprising an acid, a buffer, and an antimicrobial metal in an amount and time sufficient to reduce a microbial population.

Another aspect of the invention provides for performance of the above described methods at intermittently fluidly connected stations. Such stations include a sanitation station for contacting the seafood with the disinfection composition or the recycled disinfection composition immediately after seafood trimming. This sanitation station is connected to a holding and/or stunning apparatus, a chilling apparatus, a skinning apparatus, or a combination of these. The intermittent fluid connection allows transfer of recycled disinfection composition from the sanitization station to connected stations.

Various embodiments of the above described methods reduce odor of the seafood, delay onset of odor of the seafood, and/or extend shelf-life of the seafood. Some embodiments of the above described methods inhibit glycolytic, proteolytic, and/or lipolytic enzymatic seafood degradation.

Another aspect of the present invention is a seafood processing system with at least a holding and/or holding/stunning station; a chilling station; and sanitization station. Each of these system stations are intermittently fluidly connected via a buffered acidic disinfection composition.

In some of the various aspects and embodiments, the processed seafood can include fish, crustacean, mollusc. Preferably, the seafood being processed is fish. For example, fish that can be processed according to the methods and systems described herein include anchovy, barramundi, bass, butterfish, carp, catfish, capelin, cod, croaker, eel, flounder, flathead, flatfish, groundfish, haddock, halibut, harvestfish, hilsa, herring, John Dory, kapenta, mackerel, mahi-mahi, milkfish, monkfish, orange roughy, saury, panfish, pollock, pilchard, redfish, salmon, sardine, scrod, sea bass, seer fish, shad, shrimpfish, silver carp, skate, snapper, snook, snoek, sole, sturgeon, swordfish, tilapia, trout, tuna, turbot, walleye, walu, whitebait, whitefish, and whiting.

The disinfection composition or the recycled disinfection composition can have a pH of about 1.5 to about 6, preferable about pH 1.5 to about pH 4, more preferable about pH 2 to about pH 3, especially a pH of about 2. The disinfection composition can include sulfuric acid and ammonium sulfate and/or sodium sulfate. The antimicrobial metal of the disinfectant composition or recycled composition can be copper, zinc, magnesium, or silver. For example, the disinfection composition can include sulfuric acid, ammonium sulfate and/or sodium sulfate, and copper sulfate. Where the antimicrobial metal is copper, the copper concentration in the disinfection composition or recycled disinfection composition can be about 1 ppm to about 20 ppm (e.g., about 3 ppm). Alternatively, concentrated copper-containing disinfection composition can be added to seafood processing water in an amount sufficient to provide a copper concentration of about 1 ppm to about 20 ppm (e.g., about 3 ppm). The disinfection composition for use in the above described methods and systems can further include a stabilizing agent, wetting agent, hydrotrope, thickener, foaming agent, acidifier, pigment, dye, surfactant, or some combination thereof. The disinfection composition for use in the above described methods and systems can consist essentially of ingredients generally recognized as safe (GRAS) food additives.

Other objects and features will be in part apparent and in part pointed out hereinafter.

### DRAWINGS

Those of skill in the art will understand that the drawings, described below, are for illustrative purposes only. The drawings are not intended to limit the scope of the present teachings in any way.

FIG. 1 is a series of process flow diagrams. FIG. 1A depicts an overview of a seafood processing system. FIG. 1B depicts an alternative of a microorganism intervention system according to the present invention.

FIG. 2 is a process flow diagram depicting an alternative of a microorganism intervention system according to the present invention.

FIG. 3 is a bar graph showing the log of psychrotrophic plate count (PPC) as for control and Tasker Pacific Blue treated catfish fillets at one and seven days after application. For further methodology information, see Example 1.

FIG. 4 is a line and scatter plot showing log of aerobic plate counts (APC) reported in colony forming units (CFU) as a function of time (1, 5, and 11 days) for catfish fillets treated with Tasker Pacific Blue at pH 2 or pH 3 for 5, 10, or 15 minutes. For further methodology information, see Example 2.

FIG. 5 is a line and scatter plot showing the log of SPC as a function of time (3-10 days) for control and Tasker Pacific Blue treated salmon filets. For further methodology information, see Example 3.

FIG. 6 is a bar graph showing log aerobic plate counts (APC) for control and Tasker Pacific Blue treated scallops over time (0, 2, 5, 8, 12, and 16 days). For further methodology information, see Example 4.

FIG. 7 is a bar graph showing the log aerobic plate counts (APC) for control and Tasker Pacific Blue treated shrimp over time (2, 5, and 8 days). For further methodology information, see Example 5.

FIG. 8 is a line and scatter plot showing odor score as a function of time (11-14 days) for catfish fillets stored on Tasker Pacific Blue-containing ice. For further methodology information, see Example 6.

FIG. 9 is a bar graph showing odor score for shrimp treated with Tasker Pacific Blue at pH 3.5 and 1, 2, or 3 ppm copper (30 second dip). For further methodology information, see Example 8.

FIG. 10 is a bar graph showing odor score for shrimp treated with Tasker Pacific Blue at pH 2 and 1, 2, or 3 ppm copper (1 minute dip). For further methodology information, see Example 8.

FIG. 11 is a bar graph showing odor score for croaker treated with Tasker Pacific Blue at 2 ppm copper and pH 2 or pH 3.5 (5 minute dip). For further methodology information, see Example 8.

FIG. 12 is a bar graph showing odor score for salmon fillets treated with Tasker Pacific Blue at pH 3.5 and 1, 2, or 3 ppm copper (30 second spray). For further methodology information, see Example 8.

FIG. 13 is a bar graph showing odor score for whole snapper treated with Tasker Pacific Blue at pH 2 and 2 ppm copper (1 minutes and 5 minute dip). For further methodology information, see Example 8.

FIG. 14 is a bar graph showing odor score for whole snapper stored at 40° F. for 12 hours with ice containing Tasker Pacific Blue at pH 2 or pH 4-5 and 1, 2, or 3 ppm copper. For further methodology information, see Example 8.

FIG. 15 is a bar graph showing log Listeria monocytogenes over time (1 and 4 days) for control and Tasker Pacific Blue treated nutrient broth samples. For further methodology information, see Example 9.

FIG. 16 is a processing flowchart for raw fresh/frozen seafood product processing.

FIG. 17 is a processing flowchart for a typical seafood fillet processing.

FIG. 18 is a processing flowchart for typical shrimp processing.

FIG. 19 is a processing flowchart for typical lobster processing.

FIG. 20 is a processing flowchart for typical bottomfish processing.

FIG. 21 is a processing flowchart for typical salmon and/or halibut processing.

### DETAILED DESCRIPTION

The present invention is generally directed to methods to reduce pathogen contamination during food processing. The methods include the use of particular disinfection compositions suited for processing of food products, preferably meat processing, and more preferably seafood processing, at or during one or more processing steps. Such methods can reduce microbial contamination, reduce odor, and extend shelf-life for various seafood products.

Food Products

A food product generally includes any food substance that might require treatment with a disinfection composition or composition and that is edible with or without further preparation. Food products can include, for example, seafood, meat (e.g., red meat and pork), poultry, fruits and vegetables, eggs, egg products, ready to eat food, wheat, seeds, sprouts, seasonings, or a combination thereof. Produce generally includes food products such as fruits and vegetables and plants or plant-derived materials that are typically sold uncooked and, often, unpackaged, and that can sometimes be eaten raw.

The methods described herein can be applied to meat processing, especially seafood processing. A meat product generally includes various forms of animal flesh, including muscle, fat, organs, skin, bones, and body fluids and like components that form the animal. Animal flesh includes the flesh of mammals, birds, fishes, reptiles, amphibians, snails, clams, crustaceans, other edible species such as lobster, crab, etc., or other forms of seafood. The forms of animal flesh include, for example, the whole or part of animal flesh, alone or in combination with other ingredients. Typical forms include, for example, fresh, frozen, or marinated fish fillets, canned seafood, seafood meal (e.g., fish meal), seafood oil or seafood protein products, such as surimi, processed seafood such as cured seafood, sectioned and formed products, minced products, finely chopped products, ground seafood meat, and products including ground meat, whole products, and the like. For example, the methods of the present invention can be applied to processing of retail seafood. Such application can provide reduced microbial levels, odor knockdown, and enhanced shelf-life.

Preferably, the methods described herein are applied to seafood processing. Seafood generally includes any aquatic organism or derivative of aquatic organism that is served as food or is suitable for eating. This can include seawater animals, such as fish and shellfish (including mollusks and crustaceans), as well as similar animals from fresh water and all edible aquatic animals collectively referred to as seafood, as well as the eggs of these animals. Examples of seafood include edible fish such as American shad, American sole, anchovy, antarctic cod, arrowtooth eel, asian carp, atka mackerel, atlantic cod, atlantic eel, atlantic herring, atlantic salmon, atlantic trout, australasian salmon, black mackerel, blue cod, bluefin tuna, brook trout, butterfish, barramundi, California halibut, capelin, carp, catfish, cherry salmon, chinook salmon, chum salmon, cod, coho salmon, eel, european eel, european flounder, flathead, flatfish, flounder, freshwater eel, freshwater herring, groundfish, haddock, halibut, harvestfish, herring, hilsa, Japanese butterfish, John Dory, kapenta, lemon sole, mackerel, maori cod, mahi-mahi, milkfish, monkfish, northern anchovy, Norwegian atlantic salmon, orange roughy. pacific cod, pacific herring, pacific salmon, pacific saury, pacific trout, panfish, pelagic cod, pink salmon, pollock, pilchard, rainbow trout, redfish, red snapper, round herring, Russian sturgeon (including eggs), salmon, sardine, saury, scrod, sea bass, seer fish, shrimpfish, silver carp, skipjack tuna, sole, snook, snoek, Spanish mackerel, sturgeon (including eggs), surf sardine, swamp-eel, swordfish, striped bass, skate, tilapia, trout, tuna, turbot, walleye, walu also known as butter fish, whitebait, whitefish, whiting, and yellowfin tuna. Examples of seafood also include the edible eggs of fish, such as caviar (sturgeon roe), Ikura (salmon roe), kazunoko (herring roe), lumpfish roe, masago (capelin roe), and tobiko (flying-fish roe). Examples of seafood also include shellfish, which includes molluscs and crustaceans such as crab, particularly dungeness crab, king crab, snow crab; crayfish; lobster, particularly American lobster and rock lobster/spiny lobster; shrimp; prawns; abalone; clam; cockle; conch; cuttlefish; mussel; octopus; oyster; periwinkle; snail; squid; and scallop, specifically bay scallop and sea scallop. Seafood also includes other aquatic organisms, such as sea cucumber and Uni (sea urchin “roe”).

Seafood products can include whole, sectioned, processed, cooked or raw seafood, and encompass all forms of seafood flesh, by-products, and side products. The flesh of seafood includes muscle, fat, organs, skin, bones, and body fluids and like components that form the animal. Forms of animal flesh include, for example, the whole or part of seafood flesh, alone or in combination with other ingredients. Typical forms include, for example, processed seafood meat, such as cured seafood meat, sectioned and formed products, minced products, finely chopped products and whole products. Seafood processing methodology is well known in the art (see generally, Welt (1995) Seafood Regulations Compliance Manual, Springer, ISBN: 0412987511; Shahidi et al. (1997) Seafood Safety, Processing, and Biotechnology, CRC, ISBN: 1566765730; Haard and Simpson (2000) Seafood Enzymes: Utilization and Influence on Postharvest Seafood Quality, CRC, ISBN: 082470326X; Pigott (1990) Seafood, Food Science and Technology, CRC, ISBN: 0824779223; Bonnell (1994) Quality Assurance in Seafood Processing, Springer, ISBN: 0442008791; Hall (1997) Fish Processing Technology, Springer ISBN: 0751402737). Except as otherwise noted herein, therefore, the process of the present invention can be carried out in accordance with such processes.

Application

Food products can be contacted with the disinfection composition described herein by any method or apparatus suitable for applying the disinfection composition. For example, the disinfection composition can be delivered as a vented densified fluid composition, a spray of the agent, by immersion in the agent, by foam or gel treating with the agent, or the like, or any combination thereof. Contact with a gas, spray, foam, gel, or by immersion can be accomplished by a variety of methods known to those of skill in the art for applying agents to food.

The disinfection compositions described herein can be employed for a variety of disinfection purposes, preferably as, or for forming, water-based systems for processing and/or washing seafood. The present methods, employing the disinfectant composition described herein, can be employed for processing meat at any step from gathering the live animals through packaging the final product. For example, the present compositions and methods can be employed for washing, rinsing, or chilling seafood for reducing contamination of these items with spoilage/decay-causing bacteria, and pathogenic bacteria.

Seafood Processing

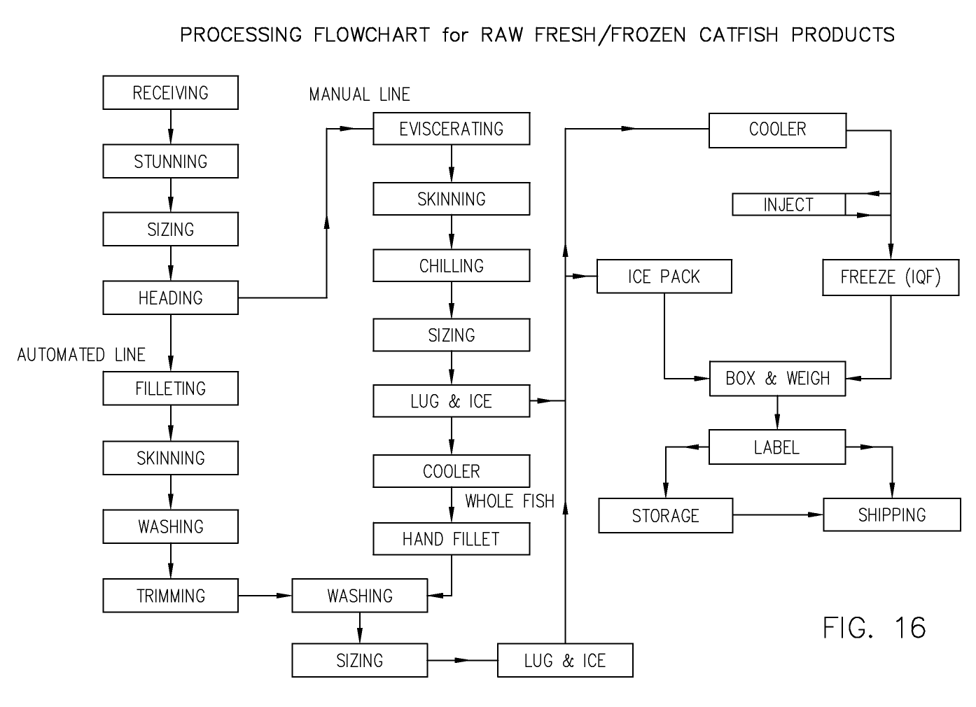

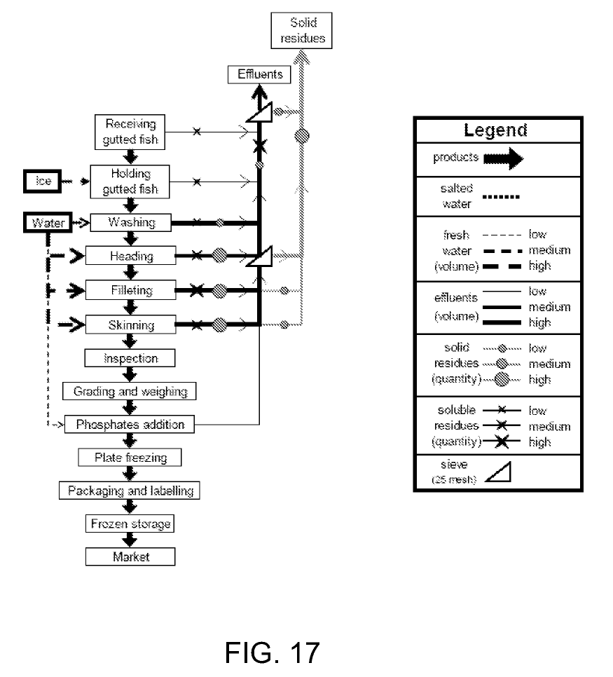

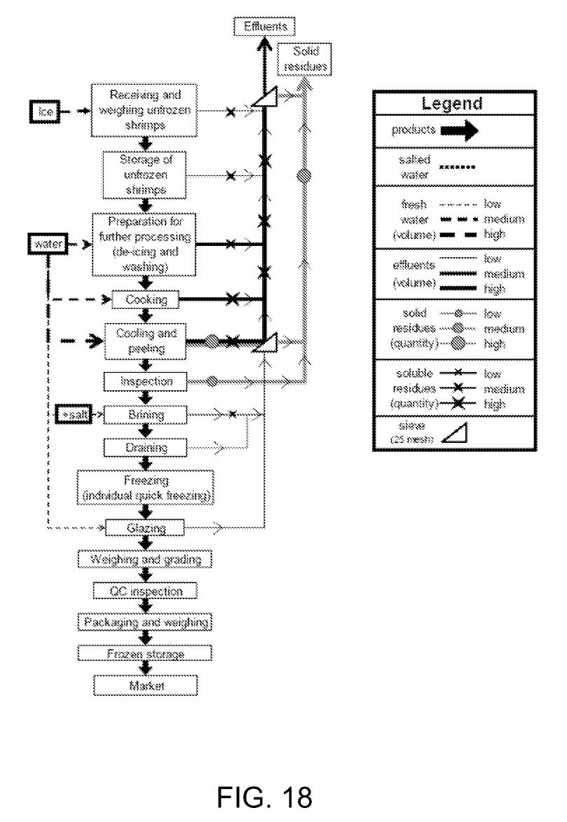

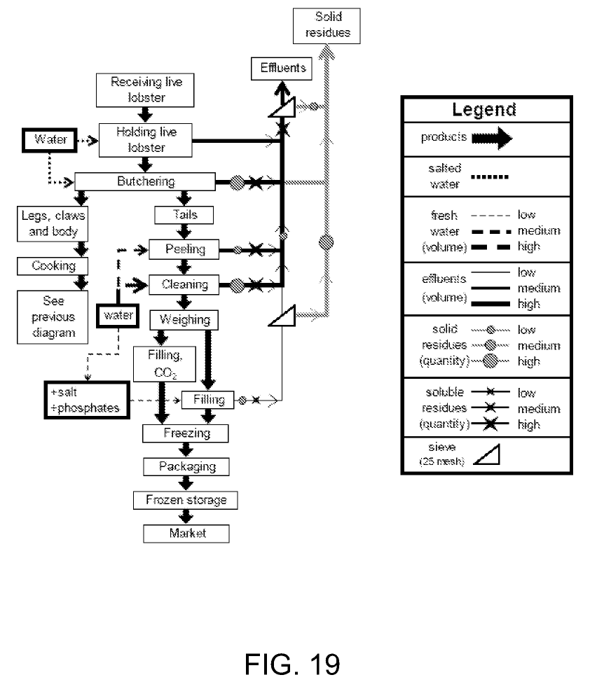

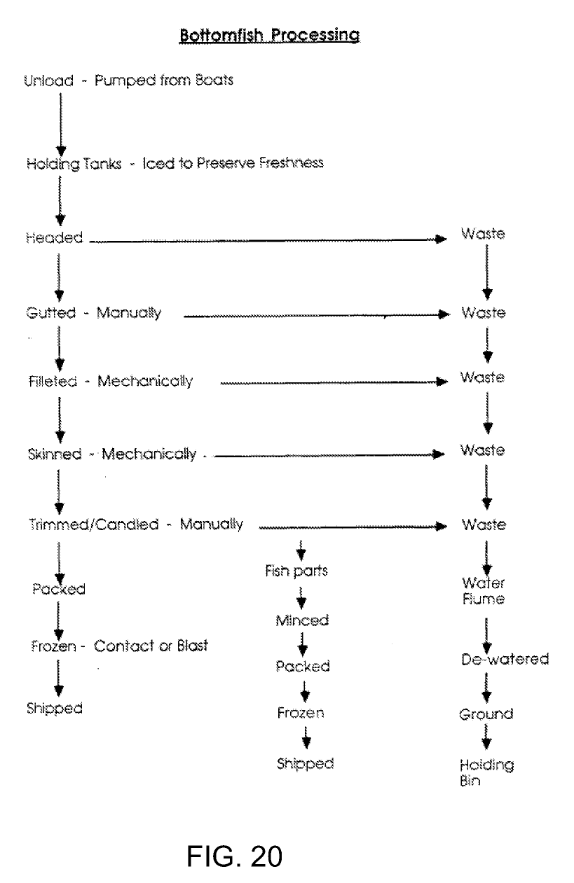

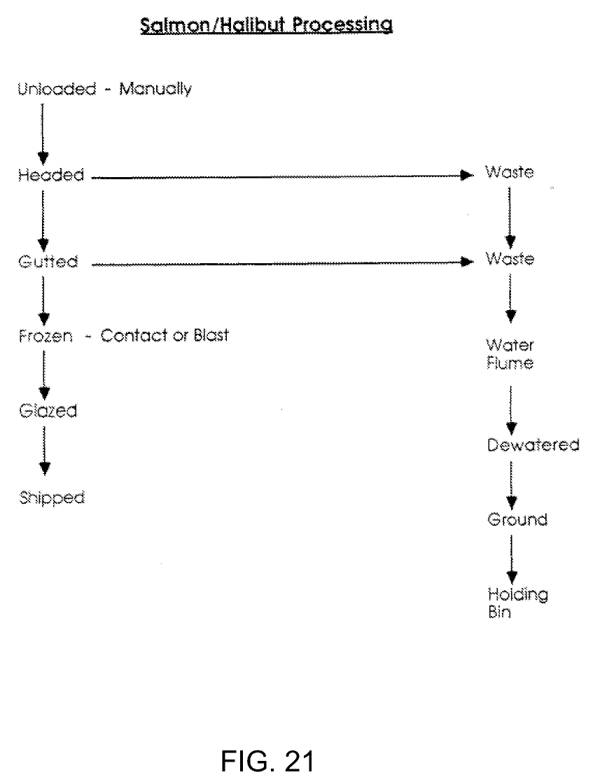

The disinfection composition of the present invention can be used at any stage of seafood harvesting and/or processing so as to reduce microbial contamination, reduce development of odor, reduce existing odor, and/or increase shelf-life. For example, disinfection composition described herein can be used in a spray, dip, slurry, or ice involved in most any stage of seafood harvesting, transport, processing, and/or storage. The following seafood processing discussion is directed generally to fish processing (see e.g., FIG. 16; FIG. 17; FIG. 20; FIG. 21), but it is within the skill of the art to adapt the processing methodology described below to other seafood, such as molluscs, crustaceans, etc. (see e.g., FIG. 18; FIG. 19).

Fresh seafood is generally harvested by boat in waterways or the open sea or from seafood farms. Seafood processing can take place on board the harvesting vessel. For example, the entire seafood processing operation, including fish meal and oil production for offal and fish waste, can take place onboard the harvesting vessel. As such, each of the steps described in more detail below can occur on the harvesting vessel. And the disinfection composition can be employed in each of these steps on the harvesting vessel as described below. But generally, upon harvesting, fresh seafood is usually stored on ice or in a slurry until transported to the processing facility. The disinfection composition described herein can be applied directly to the surface of the harvested seafood while on the harvesting vessel, for example by dipping or spraying or by inclusion in holding tanks, arctic boxes, etc. The disinfection composition can be also included in the ice (e.g., Pacific Blue Seafood Ice) upon and/or in which the harvested seafood is stored. As another example, the disinfection composition of the present invention can be employed in the transport, storage, display, and/or transfer to an end-user at a dockside seafood market.

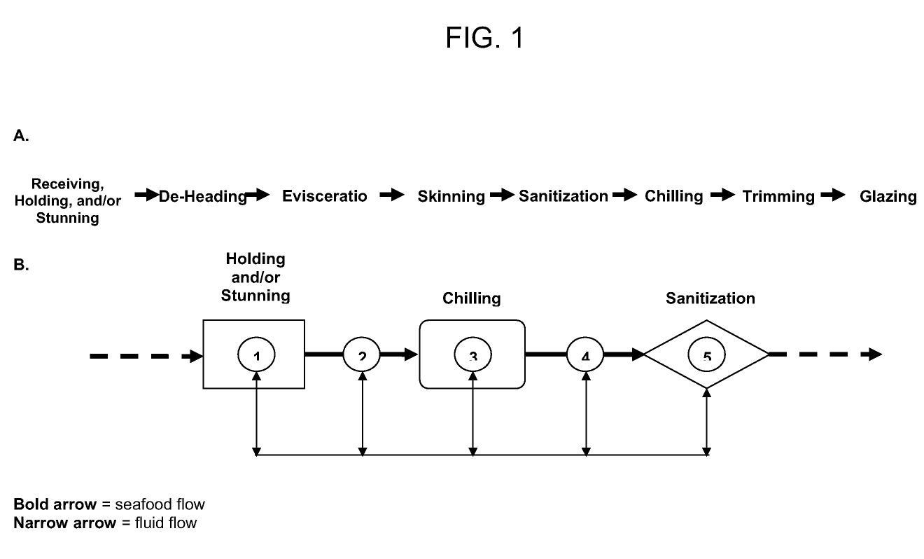

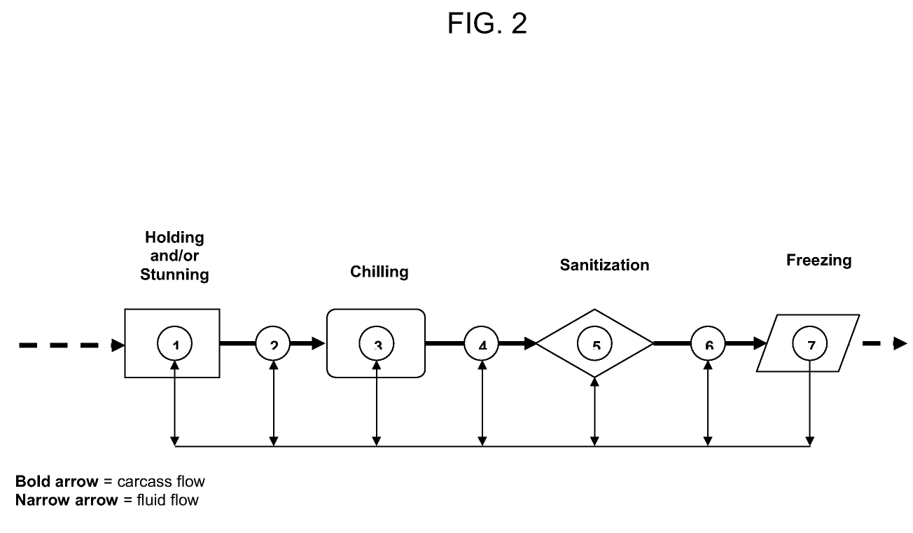

Seafood processing generally involves several steps, including receiving, holding, stunning (for farm-raised seafood), sizing, de-heading, filleting, skinning, trimming, packing, and storage; with chilling, washing, and/or rinsing also occurring before, during, and/or after these steps. These processes can take place within separate departments or stations of the seafood processing plant (see e.g., FIG. 1; FIG. 2). The disinfection composition described herein can be employed at any/all of these stages, as described in more detail below.

Fresh seafood can be transported from, for example, boat, dock, or farm to a processing facility and re-iced or placed into a holding tank which can contain an ice slurry. These tanks can contain a brine solution to help reduce bacterial loads on the whole seafood. The disinfectant composition of the present invention can be applied to the fresh seafood surface before placing in the holding tank. The disinfection composition can be added to the holding tank water, the holding tank ice, and/or the holding tank brine. Generally, in this step and others, the liquid disinfection composition in the bath can be agitated, sonicated, or pumped to increase contact of the disinfection composition with the seafood. The tank can also include one or more additional ingredients permitted in holding tanks.

Seafood product can be transferred into a stun tank where they are prepared for de-heading (if not previously de-headed). Stunning tanks are usually employed where the seafood is farm-raised. The disinfectant agent of the present invention can be applied to the fresh seafood surface before placing in the stun tank and/or the disinfection composition can be added to the stun tank water. Similarly, disinfectant agent of the present invention can be applied to the fresh seafood surface before placing in the holding tank and/or the disinfection composition can be added to the holding tank water. The stunning tank can also include one or more additional ingredients permitted in stun tanks.

The seafood can be de-headed and eviscerated (either automatically or by hand). Microbial contamination of the seafood muscle can occur during de-heading and filleting. The disinfection composition described herein can be used to wash or rinse (e.g., through dip or spray) the seafood during de-heading and/or eviscerating. The disinfection composition described herein can also be used to wash and/or rinse the equipment, conveyors, blades, and the like that come into contact with the seafood during de-heading and/or eviscerating.

After de-heading and/or evisceration, the seafood can be rinsed and/or washed, processes which generally entail spraying or dipping seafood with or in water, typically at a temperature of about 5 to about 30° C. According to the present invention, rinsing and/or washing can be accomplished employing a disinfection composition described herein. Rinsing is typically accomplished with a washing apparatus such as a wash or spray cabinet with stationary or moving spray nozzles. To increase contact with the seafood, the disinfection compositions in the spray water can be applied at higher pressures, flow rates, temperatures, or with agitation or ultrasonic energy. Alternatively, a “flood”-rinsing or liquid submersion washing apparatus can be used.

Vertebrate seafood can be filleted, where the meat is separated from the skeleton. Filleting can be performed manually or with mechanical filleting machines. A filleting department or station can be physically separated from the pretreatment area so as to prevent contamination passing from the (non-sterile) pretreatment area to the (sterile) filleting area. Filleting machines may comprise pairs of mechanically operated knives which cut the fillets from the backbone and remove the collarbone. The disinfection composition can be employed during or immediately after filleting. For example, the disinfection agent can be applied to the seafood and/or used to wash or rinse filleting equipment that comes into contact with the seafood.

Some seafood products may require skinning. Skinning can be accomplished by immersion in, for example, a warm caustic bath. The effluent generated from this process usually has a high organic load. The disinfection composition described herein can be applied to the seafood before, during, and/or after skinning. For example, the disinfection composition can be included the skinning bath. The composition of the present invention is particularly suited for efficient functioning in high temperature, high organic load environments such as a skinning bath.

The seafood can be inspected and trimmed to remove defects and parts of inferior quality. At the trimming stage, offcuts can be collected and minced and, depending on the final product, the seafood may be portioned or divided into parts such as loin, tail and belly flap. Before packaging, the seafood can be inspected to ensure they meet product standard. The disinfection composition described herein can be employed during or immediately after trimming.

Seafood is generally washed and/or rinsed after de-heading, eviscerating, filleting, and/or trimming. Seafood washing typically includes rinsing the interior and exterior surfaces of the seafood with streams or floods of water, typically at a temperature of about 5 to about 30° C. To increase contact with the seafood, the disinfection compositions in the spray water can be applied at higher pressures, flow rates, temperatures, or with agitation or ultrasonic energy. Seafood washing is generally accomplished by an apparatus that floods the seafood with streams of water in the inner cavity and over the exterior of the seafood. Such an apparatus can include a series of fixed spray nozzles to apply disinfection composition to the exterior of the seafood and a rinse probe or bayonet that enters and applies antimicrobial composition to the body cavity. According to the present invention, final washing can be accomplished employing a disinfection composition described herein (e.g., Pacific Blue Processing Wash).

Both the interior (in, for example, whole fish) and the exterior of the seafood product can be subjected to further decontamination. This further decontamination can be accomplished in part by a step commonly known as antimicrobial spray rinsing, sanitizing rinsing, or finishing rinsing. Such rinsing typically includes spraying the interior and exterior surfaces of the seafood with water, typically at a temperature of about 5 to about 30° C. To increase contact with the carcass, the disinfection compositions in the spray water can be applied using fixed or articulating nozzles, at higher pressures, flow rates, temperatures, with agitation or ultrasonic energy, or with rotary brushes. Spray rinsing is typically accomplished by an apparatus such as a spray cabinet with stationary or moving spray nozzles. The nozzles create a mist, vapor, or spray that contacts the seafood surfaces. According to the present invention, antimicrobial spray rinsing, sanitizing rinsing, and/or finishing rinsing can be accomplished employing a disinfection composition described herein.

The seafood product can be made ready for packaging or for further processing by chilling, specifically submersion chilling or air chilling. Typically, seafood products are placed either into a large cold water tank for storage (possibly containing a brine solution) or passed through a chiller (e.g., a large spiral tank with a dwell time of 15-60 minutes). Submersion chilling both washes and cools the seafood product to retain quality of the meat. Submersion chilling typically includes submersing the seafood product completely in water or slush, typically at a temperature of less than about 5° C., until the temperature of the seafood product approaches that of the water or slush. Chilling of the seafood product can be accomplished by submersion in a single bath, or in two or more stages, each of a lower temperature. Water can be applied with agitation or ultrasonic energy to increase contact with the carcass. Submersion chilling is typically accomplished by an apparatus such as a tank containing the chilling liquid with sufficient liquid depth to completely submerse the seafood product. The seafood product can be conveyed through the chiller by various mechanisms, such as an auger feed or a drag bottom conveyor. Submersion chilling can also be accomplished by tumbling the seafood product in a chilled water cascade. According to the present invention, submersion chilling can be accomplished employing a disinfection composition described herein (e.g., Pacific Blue Seafood Dip/Spray). For example, the disinfection composition can be included in the chilling tank(s). Further, the disinfection agent can be employed as a pre- or post-chill in-line dip or spray.

Like submersion chilling, air chilling or cryogenic chilling cools the seafood product to retain quality of the meat. Air cooling can be less effective for decontaminating the seafood product, as the air typically would not dissolve, suspend, or wash away contaminants. Air chilling with a gas including a disinfection composition can, however, reduce the burden of microbial, and other, contaminants on the seafood product. Air chilling typically includes enclosing the seafood product in a chamber having a temperature below about 5° C. until the seafood is chilled. Air chilling can be accomplished by applying a cryogenic fluid or a gas or a refrigerated gas as a blanket or spray. According to the present invention, air chilling or cryogenic chilling can be accomplished employing a disinfection composition described herein. For example, air chilling compositions can include a gaseous or densified fluid disinfection composition.

After chilling, the seafood product can be subjected to additional processing steps including weighing, quality grading, allocation, portioning, further deboning, and the like. This further processing can also include methods or compositions according to the present invention for washing with disinfection compositions. For example, it can be advantageous to wash seafood product portions formed by portioning the seafood carcass. Such portioning forms or reveals new meat, skin, or bone surfaces which may be subject to contamination and benefit from treatment with a disinfection composition. Washing or rinsing the further processed seafood product with a disinfection composition described herein can advantageously reduce any such contamination. In addition, during any further processing, the seafood product can also come into contact with microbes, for example, on contaminated surfaces. Washing or rinsing the seafood product (or contact surfaces) with a disinfection composition can reduce such contamination. Washing can be accomplished by spraying, immersing, tumbling, or a combination thereof, or by applying a gaseous or densified fluid disinfection composition.

The fillets can be removed from the storage tank or chiller and passed to a sizing process. Sizing can be done either visually or automatically. Typically a machine will automatically size each seafood product (e.g., fish fillet) and place them into storage vessels to achieve uniformity. The disinfection composition described herein can be employed in the sizing process via direct application to the seafood product and/or as a wash or rinse for equipment or surfaces employed during sizing. Here the seafood products are prepped either for packaging, where they are boxed and stored for distribution, or they are directed to contact/blast freezing. The disinfection composition described herein can be employed in the preparation for packaging or inside the packaging of seafood product.

For seafood products that will be frozen, the seafood products can be transferred from the storage or chiller tanks and passed through a glazer in order to coat the seafood product with water to be frozen. The disinfection composition described herein can be employed in the water glaze. The seafood product can be transferred from the glazer to a conveyer belt where it is sorted to maintain separation and moved along to either a blast freezer or a contact freezer. A contact freezer uses direct contact with the cooling system to achieve freezing whereas the blast freezer circulates super cooled air. After freezing, the seafood products are packaged and stored in a cool room until distribution. The disinfection composition described herein can be employed in the packaging process and/or included in the final packaged frozen seafood product.

The seafood product can be packaged before sending it for more processing, to another processor, into commerce, or to the consumer. Any such product can be washed with a water based disinfection composition, which may then be removed (e.g., drained, blown, or blotted) from the seafood product. In certain circumstances wetting the seafood product before packaging is disadvantageous, in which case, a gaseous or densified fluid form of the disinfection composition can be employed for reducing the microbial burden on the seafood product. Such a gaseous disinfection composition can be employed in a variety of processes known for exposing a seafood product to a gas before or during packaging, such as modified atmosphere packaging.

Fresh seafood products can be packaged in, for example, containers with ice, the ice being separated from the products by a layer of plastic. Frozen products can be packed in a number of ways. For example, seafood products can be individually frozen and wrapped in plastic, or commonly packed as 6-11 kg blocks in waxed cartons. The blocks are typically frozen and then kept in cold storage. The disinfection composition described herein can be employed during the packaging process and/or included in the packaged seafood product. For example, the disinfection composition can be applied to the seafood product before or during packaging. As another example, the disinfection composition can be included in the ice used to pack the fresh seafood product. As a further example, the disinfection composition can be used as a glaze during the freezing process.

After processing, seafood product can be stored, shipped, and/or displayed before eventual purpose by an end user/consumer. Each transfer of seafood product entails more opportunity for microbial contamination and elapsed time increases natural degradation processes. The disinfection composition described herein can be applied to the seafood product at any post-processing stage up to, and including, purchase and use by an end user (e.g., Pacific Blue Seafood Dip/Spray). For example, the disinfection composition can be employed during transport of the processed seafood product. As another example, the disinfection composition can be employed (e.g., as a dip, spray, and/or in ice) during storage and/or display of the seafood product. As a further example, the disinfection composition can be employed at the time of purchase by a merchant and/or consumer and/or during a consumer's transport, storage, and/or preparation of the seafood product. As yet a further example, the disinfection composition can be employed in the restaurant/food industry during transport, storage, and/or preparation of the seafood product.

Usable side products of seafood processing can be harvested later in processing and sold as food products. Of course, microbial contamination of such food products is undesirable. Thus, disinfection composition can be applied to these side products according to methods of the present invention. Typically, these side products will be washed after harvesting from the seafood and before packaging. They can be washed by submersion or spraying with the disinfection composition, or transported in a flume including the disinfection composition. They can be contacted with a disinfection composition according to the invention in, for example, an ice chiller.

The advantageous stability of the disinfection compositions described herein in such methods as described above, which include the presence of seafood product debris or residue, makes these compositions competitive with cheaper, less stable, and potentially toxic chlorinated compounds. Preferred methods of the present invention include agitation or sonication of a use composition, particularly as a concentrate is added to water to make the use composition. Preferred methods include water systems that have some agitation, spraying, or other mixing of the solution.

The disinfection compositions described herein can be contacted with the seafood product in an amount effective to result in a reduction significantly greater than is achieved by washing with water, or at least a 50% reduction, preferably at least a 90% reduction, more preferably at least a 99% reduction, in the resident microbial preparation.

The disinfection composition described herein can be applied to whole seafood and/or exposed meat (e.g., fish fillet). The disinfection composition may be more effective on exposed meat. Without being bound by a particular theory, it is possible that exposure of meat to the applied low pH disinfection composition of the present invention can inhibit endogenous enzymes in the meat which are associated with enzymatic degradation processes, such as glycolysis, proteolysis, and/or lypolysis. Thus, in addition to decreasing microorganism contamination on the seafood product, which can lead to shortened shelf-life, the disinfection compositions of the present invention may also inhibit endogenous shelf-life shortening enzymatic activity.

The present methods require a certain minimal contact time of the composition with food product for occurrence of significant disinfection effect. The contact time can vary with concentration of the use composition, method of applying the use composition, temperature of the use composition, amount of soil and/or contamination on the seafood product, number of microorganisms on the seafood product, type and formulation of the disinfection composition, or the like. Required exposure time for effective shelf-life extension is generally shorter than that required for processing of other meats, such as poultry. The minimum exposure time is, for example, at least about 2 to about 5 seconds. The exposure time can be, for example, at least about 5 seconds, at least about 10 seconds, at least about 15 seconds, at least about 30 seconds, at least about 45 seconds, at least about one minute, at least about two minutes, at least about three minutes, at least about four minutes, at least about five minutes, at least about six minutes, at least about seven minutes, at least about eight minutes, at least about nine minutes, at least about ten minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 60 minutes. Even longer exposure times are contemplated, for example, several hours or even days. Generally, longer exposure times to the disinfection composition will be preferred as the pH increases and/or the copper content decreases. After direct exposure during administration, it is contemplated that the disinfection composition can remain in contact with the seafood product for extended periods of time. For example, the disinfection composition (e.g., Pacific Blue Pacific Seafood Spray) can be sprayed directly on seafood for 2-5 seconds so as to cover all surfaces and, optionally, allowed to remain on the seafood.

Application of the disinfection composition to seafood products can occur in periodic sequential applications. An effective amount of disinfection composition can be applied to the seafood product several times per day. For example, the disinfection composition can be applied to part or all of the seafood product about every one hour, about every two hours, about every three hours, about every four hours, about every five hours, about every six hours, about every seven hours, about every eight hours, about every nine hours, about every ten hours, about every eleven hours, or about every twelve hours. For example, the disinfection composition (e.g., Pacific Blue Pacific Seafood Spray) can be sprayed directly on seafood so as to cover all surfaces, optionally allowing the composition to remain on the seafood, and re-applying 3-4 times daily or about every 4 hours. Longer periods of time between applications are contemplated. For example, the disinfection composition can be applied every day or every several days.

Application Means

A preferred method for seafood washing employs a pressure spray of the disinfection composition. During application of the spray solution on the seafood product, the surface of the product can be moved with mechanical action, preferably agitated, rubbed, brushed, etc. Agitation can be by physical scrubbing of the seafood product, through the action of the spray solution under pressure, through sonication, or by other methods. Agitation increases the efficacy of the spray solution in killing micro-organisms, perhaps due to better exposure of the solution into the crevasses or small colonies containing the micro-organisms. The spray solution, before application, can also be heated to a temperature of about 150 to 60° C., preferably about 20° C., to increase efficacy.

Application of the disinfection composition by spray can be accomplished using a manual spray wand application, an automatic spray of seafood product moving along a production line using multiple spray heads to ensure complete contact, a manual spray bottle, or other spray means. One preferred automatic spray application involves the use of a spray booth. The spray booth substantially confines the sprayed disinfection composition to within the parameter of the booth. For example, in seafood processing, the production line moves the seafood product through the entryway into the spray booth in which the seafood product is sprayed on all its exterior surfaces with sprays within the booth. After a complete coverage of the material and, optionally, drainage of the material from the seafood product within the booth, the seafood product can then exit the booth in a fully treated form. The spray booth can include steam jets that can be used to apply the antimicrobial compositions of the invention. These steam jets can be used in combination with cooling water to ensure that the treatment reaching the seafood product surface is less than 65° C., preferably less than 60° C. The temperature of the spray on the seafood product is important to ensure that the seafood product is not substantially altered (cooked) by the temperature of the spray. The spray pattern can be virtually any useful spray pattern.

Immersing a food product in a liquid disinfection composition can be accomplished by any of a variety of methods known to those of skill in the art. During processing, the seafood product can be immersed into a vessel containing a quantity of washing solution containing disinfection composition. The washing solution is preferably agitated to increase the efficacy of the solution and the speed in which the solution reduces micro-organisms accompanying the food product. Agitation can be obtained by conventional methods, including ultrasonics, aeration by bubbling air through the solution, by mechanical methods, such as strainers, paddles, brushes, pump driven liquid jets, or by combinations of these methods. The disinfection composition can be heated to increase the efficacy of the solution in killing micro-organisms. After the seafood product has been immersed for a time sufficient for the desired effect, the seafood product can be removed from the bath or flume and the disinfection composition can be rinsed, drained, or evaporated off the seafood product. It is preferable that the seafood product be immersed in the washing solution after the seafood product have been held, stunned, de-headed, eviscerated, filleted, and/or skinned.

In another alternative embodiment of the present invention, the food product can be treated with a foaming version of the disinfection composition. The foam can be prepared by mixing foaming surfactants with the disinfection composition at time of use. The foaming surfactants can be nonionic, anionic, or cationic in nature. Examples of useful surfactant types include, but are not limited to the following: alcohol ethoxylates, alcohol ethoxylate carboxylate, amine oxides, alkyl sulfates, alkyl ether sulfate, sulfonates, quaternary ammonium compounds, alkyl sarcosines, betaines and alkyl amides. The foaming surfactant is typically mixed at time of use with the disinfection composition or composition. At time of use, compressed air can be injected into the mixture, then applied to the seafood product surface through a foam application device such as a tank foamer or an aspirated wall mounted roamer.

In another alternative embodiment of the present invention, the seafood product can be treated with a thickened or gelled version of the disinfection composition. In the thickened or gelled state, the disinfection composition remains in contact with the seafood product surface for longer periods of time, thus increasing the antimicrobial efficacy. The thickened or gelled solution will also adhere to vertical surfaces. The composition can be thickened or gelled using existing technologies such as xanthan gum, polymeric thickeners, cellulose thickeners, or the like. Rod micelle forming systems such as amine oxides and anionic counter ions could also be used. The thickeners or gel forming agents can be used either in the concentrated product or mixed with the disinfection composition at time of use. Typical use levels of thickeners or gel agents range from about 100 ppm to about 10 wt-%.

In another alternative embodiment of the present invention, the food product can be exposed to an activating light (or other electromagnetic radiation) source following application of the disinfection composition. The activating light (or other electromagnetic radiation) can improve the efficacy of the disinfecting agent. The light can be ultraviolet light, infrared light, visible light, or a combination thereof. Other forms of electromagnetic radiation include radar and microwave.

Disinfection Composition

The disinfection compositions utilized in the methods described herein are effective for killing one or more of the food-borne pathogenic bacteria associated with meat, particularly seafood, such as Listeria monocytogenes, Escherichia coli, Vibrio parahaemolyticus, V. vulnificus, V. mimicus, V. fluvialis, V. furnissii, V. hollisae, V. cholerae, (and other Vibrio spp.), Bacillus cereus, Clostridium botulinum, Clostridium perfringens, Campylobacter jejuni, Salmonella typhimurium, Salmonella (nontyphoidal), Shigella, Staphylococcus aureus, and the like.

The disinfection compositions and methods of the present invention have activity against a wide variety of microorganisms such as Gram positive (for example, Listeria monocytogenes) and Gram negative (for example, Escherichia coli) bacteria, yeast, molds, bacterial spores, viruses, etc. The compositions and methods of the present invention, as described above, have activity against a wide variety of human pathogens. The compositions and methods can kill a wide variety of microbes on the surface of seafood or in water used for washing or processing of seafood.

In several embodiments, reducing pathogen contamination in seafood processing is accomplished by using a non-oxidizing, acidic, buffered disinfectant that functions efficiently in a high-organic load aqueous environments. The disinfectant generally operates at a low pH, for example around about pH 1 to about pH 4, preferably about pH 1 to about pH 3, or more preferably at a pH of about 2. It can be desirable for the disinfectant to be a food safe additive (GRAS). For example, the disinfectant is preferably Generally Recognized as Safe (GRAS) by the United States of America Food and Drug Administration (FDA), such designation indicating that the chemical or substance added to food is considered safe by experts, and so is exempted from the usual Federal Food, Drug, and Cosmetic Act (FFDCA) food additive tolerance requirements.

It can also be desirable that the disinfection composition function effectively at high temperatures. Such a disinfectant can be utilized in several target steps of seafood processing, such as in receiving, holding, stunning, de-heading, eviscerating, filleting, skinning, trimming, chilling, washing, icing, freezing, storing, transporting, and/or displaying.

The preferred compositions include concentrate disinfection compositions and use disinfection compositions. Typically, a disinfection concentrate composition can be diluted, for example with water, to form a disinfection use composition. For example, the disinfection composition can be formulated such that one or several fluid ounces of concentrated disinfection composition can form one, two, three, four, five, or more gallons of use disinfection composition. In a preferred embodiment, the concentrate composition is diluted into water employed for processing seafood. The use disinfection composition can also be supplied in a storage and/or application vessel at an effective use concentration. For example, the disinfection composition for use in the methods described herein can be contained in a spray bottle.

Disinfectants within the scope of the invention include multiple-component disinfection compositions. In one embodiment, the multiple-component disinfection composition is a buffered acidic disinfection composition. The disinfection composition can be a buffered acidic solution of a strong acid and a salt of a strong acid and strong base (e.g., Tasker Clear; see Example 12). Exemplary acidic agents include those provided in Table 1. Exemplary buffering systems include corresponding salts.

For example, a buffered acidic disinfection composition for use in the methods described herein can be formed by reacting 98% sulfuric acid with a 13-18% by weight ammonium sulfate in water solution (order of addition is ammonium sulfate solution to sulfuric acid) at approximately 300-350° F. for 24 hours, where electrolysis of the reacting solution is applied for 1 hour at the start of the process, with a stabilization step (addition of more ammonium sulfate solution to ensure that the reaction is complete) after overnight cooling. As another example, the same process can be performed but at approximately 200-210° F. for 2 hours with a stabilization step immediately after the 1 hour electrolysis period. As a further example, a buffered acidic disinfection composition for use in the methods described herein can be formed, in a “cold process”, by adding 98% sulfuric acid slowly to a 30% by weight ammonium sulfate solution, with no stabilization step, at a temperature of 150-200° F. during the addition process. As yet another example, a buffered acidic disinfection composition for use in the methods described herein can be formed by reacting 8% sulfuric acid with a 13-18% by weight sodium sulfate in water (order of addition is sodium sulfate solution to sulfuric acid) for 4 hours at approximately 300-350° F. with a stabilization step (addition of more sodium sulfate solution to ensure that the reaction is complete) after cooling, where electrolysis of the reacting solution is applied for 1 hour at the start of the process. In still another example, a buffered acidic disinfection composition for use in the methods described herein can be formed, in a “cold process” (i.e., no electrolysis step), by reacting 98% sulfuric acid with a 26-28% by weight sodium sulfate in water solution for 4 hours at approximately 300-350° F. with a stabilization step after cooling.

In another embodiment, the multiple-component disinfection composition is a buffered acidic agent in combination with an antimicrobial metal-containing agent capable of providing free metal ions in solution (see U.S. application Ser. No. 11/065,678, incorporated herein by reference). Examples of such antimicrobial metals include copper, zinc, magnesium, and silver. Preferably, the multiple-component disinfection composition is a buffered acidic agent in combination with a copper containing agent capable of providing free copper ions in solution. Examples of various copper-containing agents include copper metal (inorganic copper), cuprous sulfate, cupric sulfate, and copper sulfate pentahydrate. The copper-containing buffered acidic disinfection composition for use in the methods described herein can be formed by the addition of various forms of copper to the various forms of acidic buffered disinfection composition described above.

In yet another embodiment, the multiple-component disinfection composition is an acidic agent in combination with a buffer, a sulfate-containing agent, and a antimicrobial metal agent (preferably copper). In some embodiments, a single agent can deliver both metal ions and sulfate, for example copper sulfate. Such a mixture produces a copper sulfate complex that is highly protonated and at a low pH. Further, the sulfate component is thought to enhance antimicrobial metal and proton uptake by microbes. For example, a copper-containing buffered acidic disinfection composition, also containing sulfate, can be formed by mixing water (about 68%), one of the acidic buffered disinfection compositions described above (about 12%), and copper sulfate or copper sulfate pentahydrate (about 20%) (e.g., Tasker Blue and Tasker Pacific Blue). This low pH (buffered inorganic acidic) solution serves as the active (e.g., ionic Cu2+ form) carrier of copper.

The various copper-containing buffered acidic disinfection compositions

(e.g., Tasker Blue and Tasker Pacific Blue) can be used in combination with additional buffered acidic disinfection compositions (e.g., Tasker Clear; see Example 12) to achieve the prescribed pH control and copper content of the disinfection composition. For example, the Tasker Clear™ product can be used for pH control, while the Tasker Blue or Tasker Pacific Blue product can be used for copper control—these products can be added separately or in a pre-formulated blend of Clear™ and Blue or Pacific Blue or to water to achieve the desired pH range (e.g., pH 1.5-4) and the desired copper range (e.g., 1-20 ppm). Water testing can be performed to determine the concentrations of Clear™ and Blue or Pacific Blue to add to achieve the desired targets.

It can be desirable that each of the disinfection composition ingredients are generally recognized as safe (GRAS) and are permitted for use as direct human food ingredients using good manufacturing practice.

Disinfectants described above can be produced in accord with the methods and formulations as described in U.S. patent application Ser. No. 10/922,604 (published as US 2005-0191394 A1); U.S. patent application Ser. No. 11/065,678 (published as US 2005-0191365 A1); U.S. Pat. No. 5,989,595; and U.S. Pat. No. 6,242,011 B1, each of which are incorporated herein by reference. Generally, an effective acidic copper containing disinfectant agent can be made by combining an acid, a buffer, and a copper containing substance so as to reach a pH of about 1 to about 4 and a copper concentration of about 1 ppm to about 20 ppm, preferably about 3 ppm. For example, an acid, a buffer, and a copper containing substance can be combined in equal measure in a vessel at room temperature so as to reach a pH of about 2 and a copper concentration of about 3 ppm.

TABLE 1
Acids Generally Recognized as Safe (GRAS)

| Acid Name | CAS No. |
|---|---|
| ACETIC ACID | 000064-19-7 |
| ACONITIC ACID | 000499-12-7 |
| ADIPIC ACID | 000124-04-9 |
| ALGINIC ACID | 009005-32-7 |
| P-AMINOBENZOIC ACID | 000150-13-0 |
| AMINO TRI(METHYLENE PHOSPHONIC ACID), SODIUM SALT | 020592-85-2 |
| ANISIC ACID | 001335-08-6 |
| ASCORBIC ACID | 000050-81-7 |
| L-ASPARTIC ACID | 000056-84-8 |
| BENZOIC ACID | 000065-85-0 |
| N-BENZOYLANTHRANILIC ACID | 000579-93-1 |
| BORIC ACID | 010043-35-3 |
| (E)-2-BUTENOIC ACID | 003724-65-0 |
| BUTYRIC ACID | 000107-92-6 |
| CHOLIC ACID | 000081-25-4 |
| CINNAMIC ACID | 000621-82-9 |
| CITRIC ACID | 000077-92-9 |
| CYCLOHEXANEACETIC ACID | 005292-21-7 |
| CYCLOHEXANECARBOXYLIC ACID | 000098-89-5 |
| DECANOIC ACID | 000334-48-5 |
| 5-DECENOIC ACID | 085392-03-6 |
| 6-DECENOIC ACID | 085392-04-7 |
| 9-DECENOIC ACID | 014436-32-9 |
| (E)-2-DECENOIC ACID | 000334-49-6 |
| 4-DECENOIC ACID | 026303-90-2 |
| DEHYDROACETIC ACID | 000520-45-6 |
| DESOXYCHOLIC ACID | 000083-44-3 |
| 2,4-DIHYDROXYBENZOIC ACID | 000089-86-1 |
| 3,7-DIMETHYL-6-OCTENOIC ACID | 000502-47-6 |
| 2,4-DIMETHYL-2-PENTENOIC ACID | 066634-97-7 |
| ERYTHORBIC ACID | 000089-65-6 |
| 2-ETHYLBUTYRIC ACID | 000088-09-5 |
| 4-ETHYLOCTANOIC ACID | 016493-80-4 |
| FOLIC ACID | 000059-30-3 |
| FORMIC ACID | 000064-18-6 |
| FUMARIC ACID | 000110-17-8 |
| GERANIC ACID | 000459-80-3 |
| GIBBERELLIC ACID | 977136-81-4 |
| D-GLUCONIC ACID | 000526-95-4 |
| L-GLUTAMIC ACID | 000056-86-0 |
| GLUTAMIC ACID HYDROCHLORIDE | 000138-15-8 |
| GLYCOCHOLIC ACID | 000475-31-0 |
| HEPTANOIC ACID | 000111-14-8 |
| (E)-2-HEPTENOIC ACID | 018999-28-5 |
| HEXANOIC ACID | 000142-62-1 |
| TRANS-2-HEXENOIC ACID | 013419-69-7 |
| 3-HEXENOIC ACID | 004219-24-3 |
| HYDROCHLORIC ACID | 007647-01-0 |
| 4-HYDROXYBENZOIC ACID | 000099-96-7 |
| 4-HYDROXYBUTANOIC ACID LACTONE | 000096-48-0 |
| 4-HYDROXY-2-BUTENOIC ACID GAMMA-LACTONE | 000497-23-4 |
| 5-HYDROXY-2,4-DECADIENOIC ACID DELTA-LACTONE | 027593-23-3 |
| 5-HYDROXY-2-DECENOIC ACID DELTA-LACTONE | 051154-96-2 |
| 5-HYDROXY-7-DECENOIC ACID DELTA-LACTONE | 025524-95-2 |
| 4-HYDROXY-2,3-DIMETHYL-2,4-NONADIENOIC ACID | 000774-64-1 |
| GAMMA LACTONE | |
| 6-HYDROXY-3,7-DIMETHYLOCTANOIC ACID LACTONE | 000499-54-7 |
| (Z)-4-HYDROXY-6-DODECENOIC ACID LACTONE | 018679-18-0 |
| 5-HYDROXY-2-DODECENOIC ACID LACTONE | 016400-72-9 |
| 1-HYDROXYETHYLIDENE-1,1-DIPHOSPHONIC ACID | 002809-21-4 |
| 2-(2-HYDROXY-4-METHYL-3-CYCLOHEXENYL)PROPIONIC ACID | 057743-63-2 |
| GAMMA-LACTONE | |
| 4-HYDROXY-4-METHYL-7-CIS-DECANOIC ACID | 070851-61-5 |
| GAMMALACTONE | |
| 5-HYDROXY-4-METHYLHEXANOIC ACID DELTA-LACTONE | 010413-18-0 |
| 4-HYDROXY-4-METHYL-5-HEXENOIC ACID GAMMA | 001073-11-6 |
| LACTONE | |
| 4-HYDROXY-3-METHYLOCTANOIC ACID LACTONE | 039212-23-2 |
| HYDROXYNONANOIC ACID, DELTA-LACTONE | 003301-94-8 |
| 3-HYDROXY-2-OXOPROPIONIC ACID | 001113-60-6 |
| 4-HYDROXY-3-PENTENOIC ACID LACTONE | 000591-12-8 |
| 5-HYDROXYUNDECANOIC ACID LACTONE | 000710-04-3 |
| 5-HYDROXY-8-UNDECENOIC ACID DELTA-LACTONE | 068959-28-4 |
| ISOBUTYRIC ACID | 000079-31-2 |
| ISOVALERIC ACID | 000503-74-2 |
| ALPHA-KETOBUTYRIC ACID | 000600-18-0 |
| LACTIC ACID | 000050-21-5 |
| LAURIC ACID | 000143-07-7 |
| LEVULINIC ACID | 000123-76-2 |
| LIGNOSULFONIC ACID | 008062-15-5 |
| LINOLEIC ACID | 000060-33-3 |
| L-MALIC ACID | 000097-67-6 |
| MALIC ACID | 000617-48-1 |
| 2-MERCAPTOPROPIONIC ACID | 000079-42-5 |
| 2-METHOXYBENZOIC ACID | 000579-75-9 |
| 3-METHOXYBENZOIC ACID | 000586-38-9 |
| 4-METHOXYBENZOIC ACID | 000100-09-4 |
| TRANS-2-METHYL-2-BUTENOIC ACID | 000080-59-1 |
| 2-METHYLBUTYRIC ACID | 000116-53-0 |
| 3-METHYLCROTONIC ACID | 000541-47-9 |
| 2-METHYLHEPTANOIC ACID | 001188-02-9 |
| 2-METHYLHEXANOIC ACID | 004536-23-6 |
| 5-METHYLHEXANOIC ACID | 000628-46-6 |
| 4-METHYLNONANOIC ACID | 045019-28-1 |
| 4-METHYLOCTANOIC ACID | 054947-74-9 |
| 3-METHYL-2-OXOBUTANOIC ACID | 000759-05-7 |
| 3-METHYL-2-OXOPENTANOIC ACID | 001460-34-0 |
| 4-METHYL-2-OXOPENTANOIC ACID | 000816-66-0 |
| 3-METHYLPENTANOIC ACID | 000105-43-1 |
| 4-METHYLPENTANOIC ACID | 000646-07-1 |
| 2-METHYL-2-PENTENOIC ACID | 003142-72-1 |
| 2-METHYL-3-PENTENOIC ACID | 037674-63-8 |
| 2-METHYL-4-PENTENOIC ACID | 001575-74-2 |
| 4-METHYLPENT-2-ENOIC ACID | 010321-71-8 |
| 3-METHYL-3-PHENYL GLYCIDIC ACID ETHYL ESTER | 000077-83-8 |
| 4-(METHYLTHIO)-2-OXOBUTANOIC ACID | 000583-92-6 |
| 2-METHYLVALERIC ACID | 000097-61-0 |
| MYRISTIC ACID | 000544-63-8 |
| NONANOIC ACID | 000112-05-0 |
| (E)-2-NONENOIC ACID | 014812-03-4 |
| 2-NONENOIC ACID GAMMA-LACTONE | 021963-26-8 |
| 9,12-OCTADECADIENOIC ACID (48%) AND 9,12,15- | 977043-76-7 |
| OCTADECATRIENOIC ACID (52%) | |
| OCTANOIC ACID | 000124-07-2 |
| (E)-2-OCTENOIC ACID | 001871-67-6 |
| OLEIC ACID | 000112-80-1 |
| 3-OXODECANOIC ACID GLYCERIDE | 128331-45-3 |
| 3-OXODODECANOIC ACID GLYCERIDE | 128362-26-5 |
| 3-OXOHEXADECANOIC ACID GLYCERIDE | 128331-46-4 |
| 3-OXOHEXANOIC ACID DIGLYCERIDE | 977148-06-3 |
| 3-OXOOCTANOIC ACID GLYCERIDE | 128331-48-6 |
| 2-OXOPENTANEDIOIC ACID | 000328-50-7 |
| 2-OXO-3-PHENYLPROPIONIC ACID | 000156-06-9 |
| 3-OXOTETRADECANOIC ACID GLYCERIDE | 128331-49-7 |
| PALMITIC ACID | 000057-10-3 |
| 4-PENTENOIC ACID | 000591-80-0 |
| 2-PENTENOIC ACID | 013991-37-2 |
| PERACETIC ACID | 000079-21-0 |
| PERIODIC ACID | 010450-60-9 |
| PHENOXYACETIC ACID | 000122-59-8 |
| PHENYLACETIC ACID | 000103-82-2 |
| 3-PHENYLPROPIONIC ACID | 000501-52-0 |
| PHOSPHORIC ACID | 007664-38-2 |
| POLY(ACRYLIC ACID-CO-HYPOPHOSPHITE), SODIUM | 071050-62-9 |
| SALT | |
| POLYACRYLIC ACID, SODIUM SALT | 009003-04-7 |
| POLYMALEIC ACID | 026099-09-2 |
| POLYMALEIC ACID, SODIUM SALT | 030915-61-8 |
| POTASSIUM ACID PYROPHOSPHATE | 014691-84-0 |
| POTASSIUM ACID TARTRATE | 000868-14-4 |
| PROPIONIC ACID | 000079-09-4 |
| 2-(4-METHYL-2-HYDROXYPHENYL)PROPIONIC ACID- | 065817-24-5 |
| GAMMA-LACTONE | |
| PYROLIGNEOUS ACID | 008030-97-5 |
| PYRUVIC ACID | 000127-17-3 |
| SALICYLIC ACID | 000069-72-7 |
| SODIUM ACID PYROPHOSPHATE | 007758-16-9 |
| SODIUM BISULFATE (SODIUM ACID SULFATE) | |
| SORBIC ACID | 000110-44-1 |
| STEARIC ACID | 000057-11-4 |
| SUCCINIC ACID | 000110-15-6 |
| SULFAMIC ACID | 005329-14-6 |
| SULFURIC ACID | 007664-93-9 |
| SULFUROUS ACID | 007782-99-2 |
| TANNIC ACID | 001401-55-4 |
| TARTARIC ACID, L | 000087-69-4 |
| TAUROCHOLIC ACID | 000081-24-3 |
| 1,2,5,6-TETRAHYDROCUMINIC ACID | 056424-87-4 |
| THIOACETIC ACID | 000507-09-5 |
| THIODIPROPIONIC ACID | 000111-17-1 |
| TRIFLUOROMETHANE SULFONIC ACID | 001493-13-6 |
| (2,6,6-TRIMETHYL-2- | 015356-74-8 |
| HYDROXYCYCLOHEXYLIDENE)ACETIC ACID GAMMA- | |
| LACTONE | |
| UNDECANOIC ACID | 000112-37-8 |
| 10-UNDECENOIC ACID | 000112-38-9 |
| N-UNDECYLBENZENESULFONIC ACID | 050854-94-9 |
| VALERIC ACID | 000109-52-4 |
| VANILLIC ACID | 000121-34-6 |

Generally, the longer the contact time with the seafood surface, the higher the pH should be in order to minimize organoleptic damage. Conversely, shorter contact times allow a lower pH for better microbial reductions. For example, depending upon the contact time, the pH can be about 1.0, about 1.5, about 2.0, about 2.5, about 3.0, about 3.5, or about 4.0. Each application dosage is a function of effectiveness and cost. As the pH is a logarithmic scale, nearly 10 times more disinfectant is required to reach a pH of 2.0 as needed to reach a pH of 3.0.

Where the disinfection composition comprises an acidic buffered disinfection composition, the actual application requirement is generally a function of the alkalinity of the processing plant water. The disinfectant composition can be titrated until reaching the target pH, then monitored and maintained.

In those embodiments containing a buffered acid and an antimicrobial metal, the actual application requirement is generally a function of the desired target pH and the desired metal concentration. Preferably, the seafood processing solution (e.g., rinse, chill, wash, bath, or glaze solution) contains an amount of added disinfection composition containing acid, buffer, and copper (e.g., Tasker Pacific Blue) so as to reach a pH of about 1.5 to about 4.0 and a copper content of about 2 ppm to about 20 ppm. The pH can be adjusted independently by further addition of a disinfection composition containing acid and buffer (e.g., Tasker Clear™).

Generally, the effectiveness of copper is highest at low pH; as the pH rises, the copper becomes bound and less effective. Preferably, the active, unbound copper concentration is about 1.5 ppm to about 3.5 ppm, more preferably from about 2 ppm to about 3 ppm. To counter the risk of copper being bound, the disinfectant can be added up to about 20 ppm. Concentrations above this level should be avoided so as to minimize the risk of leaving residues on the seafood product. For example, depending on the pH, contact time, and the risk of copper being bound, the copper content of the processing water can be about 1 ppm, about 1.5 ppm, about 2 ppm, about 2.5 ppm, about 3 ppm, about 3.5 ppm, about 4 ppm, about 4.5 ppm, about 5 ppm, about 5.5 ppm, about 6 ppm, about 7 ppm, about 8 ppm, about 10 ppm, about 12 ppm, about 14 ppm, about 16 ppm, about 18 ppm, or about 20 ppm. For example, disinfectant can be added to the seafood processing water so as to reach a pH of 2.0 and a copper content of 3 ppm.

As a further example, the disinfection composition or the seafood processing water containing disinfection composition can be about 98-99% water; about 0.1-0.5% copper sulfate; about 0.1-0.5% sulfuric acid; and about 0.1-0.5% ammonium sulfate, with a pH of about 2 to about 3. In yet another example, the disinfection composition or the seafood processing water containing disinfection composition can be about 98-99.9% water; about 0.001-0.01% copper sulfate; about 0.05-0.5% sulfuric acid; and about 0.01-0.1% ammonium sulfate, with a pH of about 2 to about 3, preferably with a pH of about 2 (e.g., Tasker Pacific Blue Seafood Wash 100). Such compositions can have a specific gravity at 25° C. of 1.002 or approximately 1.002; a boiling point of 212° F. or approximately 212° F.; and a freezing point of 32° F. or approximately 32° F.

The acidic buffered metal-containing disinfectants (especially the copper and sulfate containing formulations) are very effective at low concentrations and short exposure times. An effective killing dose is usually measured as concentration×time (D=C×T). Generally, antimicrobial chemicals are used at concentrations in the 10's to 100's of ppm up to a full percentage range, and often for many minutes up to several hours, in order to be effective. The effective dose of the various above disinfectant compositions for antimicrobial effect is much lower.

It is known that sulfate (SO₄²⁻), copper (Cu²⁺), and the ammonium ion (NH⁴⁺) are used by bacteria as part of their normal nutritional requirements. It is also known that at high concentrations copper sulfate can be used for plant disease control, as it is an effective antifungal agent, and will also control algae growth in lakes and ponds. Without being bound by a particular theory, the presence of sulfate, copper, and the low pH due to the presence of sulfuric acid is thought to provide various embodiments of the above disinfectant their antimicrobial properties.

The acidic buffered copper-containing disinfectant described herein is non-oxidizing. This is in sharp contrast to other antimicrobial chemicals, such as chlorine compounds, ozone, and peracetic acid. Because it is non-oxidizing the disinfectant can be used in water based environments, such as the chill tank used in seafood processing, where there is a significant amount of suspended or dissolved organic matter, without its effectiveness being impaired. Also, it will not produce oxidized compounds that will impart off-odors and flavors to the product or create toxic by-products such as tri-halomethanes (THM's). And, it will not cause the corrosion to plant and equipment typical of oxidizing chemicals.

Antimicrobial chemicals that are non-oxidizing are usually organic acids, such as lactic acid, or a combination of acids. Unlike the disinfectant compositions described herein, organic acids are usually only effective at high concentrations, creating low pH environments where the organic acid molecules are in their undissociated, non-ionize state. In this form and concentration, the organic acid can pass through the cell membrane and gain entrance into the cell. Once inside the cell, the naturally higher pH of the cell will cause the acid to ionize and release protons (hydrogen ions). This will lower the internal pH of the cell. As cellular processes will only function optimally with the internal pH in a narrow range close to neutrality (pH 7), internal “proton pumps” are used to remove the unwanted protons from the cell. This process requires the use of energy (ATP). Bacterial cell growth therefore becomes inhibited due to a depletion of cellular ATP and reduced metabolic activity, as long as it remains in a low pH environment and in the presence of these organic acids.

Without being bound by a particular theory, the following is the currently understood mechanism of action for the acidic buffered sulfate- and copper-containing disinfectant composition. Such mechanistic explanation is not intended in any way to limit the invention described herein. It is known that sulfate is required for growth of the microbial cell. It provides the cell's requirement for sulfur for the formation of the sulfur containing amino acids cysteine, cystine, and methionine, which in turn are required for the synthesis of structural and enzymatic proteins. The bacteria have a well understood process that actively transports sulfate into the cell. Thus, in a complex environment, bacterial cells will scavenge for sulfate in order to grow. The disinfectant containing buffered acid, sulfate, and copper exploits the sulfate ion scavenging function of bacterial cells.

The sulfate of the multi-component disinfectant is transported into the cell via the sulfate transport pathway and is thought to carry with it protons and copper ions. Once inside the cell, the protons are released and have to be removed via the energy consuming proton pump. In addition, the excess copper is now made available to bind to disulphide (—S—S—) and/or sulphydryl groups (—SH) associated with proteins. Interference with these groups can denature the proteins and destroy their structural or enzymatic activities, leading to the inhibition of cellular processes. Thus, there are several anti-microbial activities working in concert leading to the death of the cell: a depletion of ATP required for the removal of protons and the inactivation of structural and enzymatic proteins required for molecular synthesis.

Amount Applied

In various embodiments, contacting the disinfection composition with the food product is accomplished with a quantity of disinfection composition sufficient to acceptably reduce the microbial burden in one or more stages of seafood processing and/or inhibit the endogenous enzymatic activity associated with meat spoilage. In certain embodiments, contacting the disinfection composition with the seafood product at several stages of processing produces enhanced and/or synergistic reduction in microbial burden on the food product. The level of disinfection composition required for a desired effect can be determined by any of several methods. For example, seafood product samples can each be exposed to different amounts of disinfection composition. Then the seafood product samples can be evaluated for the amount of disinfection composition that yields the desired antimicrobial effect, and, preferably, for desired organoleptic qualities. The amount of disinfection composition required for antimicrobial effect at each processing stage can be reduced by application at several stages. Such a titration with disinfection composition can be conducted at several amounts or treatment times in combination with treatment or exposure at other stages of processing, yielding a matrix of treatment results. Such a matrix can yield a quantitative assessment of the amount of antimicrobial treatment required at various stages of processing to achieve a desired antimicrobial effect, and, optionally, desired organoleptic qualities. Synergy can be evaluated from such matrices using methods known to those of skill in the art.

The concentration of various disinfection composition can be as discussed above. Alternatively, the amount of disinfection composition added to seafood processing water can be that required to reduce microbial levels to those approved by the Food and Drug Administration for the type of seafood, or some fraction thereof (e.g., about 50%-95%). As an example, the amount of disinfection composition contacted with seafood can be that amount required to reduce microbial levels on the seafood to 95%, 90%, 85%, 80%, 75%, 70%, 65%, 60%, 55%, or 50%, or less, of the maximal amount approved by the Food and Drug Administration.

Processing Seafood Wash Water

Washing seafood products can employ a large volume of water, or another carrier. Seafood wash water can be used more than once (recycled), provided the water can be treated so that it does not transfer undesirable microbes to the seafood being washed with the recycled wash water. One way to prevent the transfer of such undesirable microbes is to reduce the microbial burden of the recycled wash water by adding one or more disinfection compositions described herein. For example, if the fluid to be recycled is water-based and lacking any disinfection composition, a disinfection composition concentrate composition can be added to result in an effective antimicrobial concentration in the fluid to be recycled. Alternatively, if the fluid to be recycled already includes or has included a disinfection composition, a disinfection composition concentrate composition can be added to increase any concentration of disinfection composition to an effective antimicrobial level. It may be that the disinfection composition in the solution to be recycled has been totally depleted, in which case more of the disinfection composition is added.

In some circumstances, the water to be recycled includes a substantial burden of organic matter or microbes. If this is the case, the water may be unsuitable for direct recycling. However, if the water is to be recycled, a sufficient quantity of the disinfection composition can be added to provide an effective antimicrobial amount of the disinfection composition after a certain amount is consumed by the organic burden or microbes already present. Then, the recycled fluid can be used with disinfection effect. Routine testing can be employed for determining levels of disinfection composition, or of organic burden.

In the case of seafood processing, the method of recycling the seafood wash water includes recovering the seafood wash water, adding a disinfection composition, and reusing the seafood wash water for processing seafood, for example, as described above. The seafood wash water can be recovered from steps in seafood processing including receiving, holding, stunning, de-heading, eviscerating, skinning, chilling, washing, glazing, freezing, storing, transporting, and/or displaying. Methods of recovering processing water from these steps are well-known to those skilled in the seafood processing arts. The wash water can also be strained, filtered, diluted, or otherwise cleaned or processed during recycling. These steps can be modified for the corresponding steps for the processing of other meat products.

FIG. 1A provides an overview of a seafood processing system, with examples of steps in which intervention with the disinfection composition described herein can be employed.

FIG. 1B depicts another alternative of a microorganism intervention system according to the present invention. Stations 1-5 are points where microbial intervention can occur both individually and in combination with other stations. A disinfection composition used in the sanitization station (shown as station 5) can be reused in the holding/stunning station (station 1), the chilling station (station 3), and intermediate stations (stations 2 and 4). For example, station 2 can be an evisceration and/or skinning station, while station 5 can be a trimming station. A disinfectant composition used at station 5 can also be reused at station 5. The bold arrows show the direction of seafood product through the system. The narrow arrows show the flow direction of an disinfection composition through the system. Bidirectional arrows depict a flow direction which can be reversible or circular. Dashed arrows depict that the same or different processes can occur before station 1 and after station 5.

FIG. 2 depicts another alternative of a microorganism intervention system according to the present invention. Stations 1-7 are points where microbial intervention can occur both individually and in combination with other stations. A disinfection composition used in the sanitization station (shown as station 5) can be reused in the holding and/or stunning station (station 1), the chilling station (station 3), and intermediate stations (stations 2 and 4). For example, station 2 can be an evisceration and/or skinning station, while station 4 can be a trimming station. A disinfection composition used at station 5 can also be reused at station 5. Similarly, a disinfection composition used at the freezing station (station 7) (e.g., as a glaze) can be reused at previous stations. The arrows show the flow direction of a disinfection composition through the system. Bidirectional arrows depict a flow direction which can be reversible or circular. Dashed arrows depict that the same or different processes can occur before station 1 and after station 7.

In one embodiment, the disinfection composition can be a liquid which can be applied by spraying on a seafood product. Excess disinfection composition can be removed from the seafood product, e.g., by falling due to gravity, and the excess can be collected followed by distribution to stations by suitable means, e.g., pumping. In an alternative, the excess disinfection composition can be distributed to the station from which it was collected and sprayed on the same or another seafood product. In certain embodiments where one or more stations are enclosed or partially enclosed, the excess disinfection composition can be collected through at least one opening in or near the bottom of station. The excess disinfection composition can then be distributed to stations by suitable means, e.g., pumping. In yet another embodiment, the station from which the disinfection composition is collected can be elevated above one or more of the stations to which the disinfectant composition is redistributed. Excess disinfection composition can fall by gravity from the seafood product directly onto one or more of the lower stations. In another embodiment, the station from which the disinfection composition is collected can be elevated above one or more of other stations, and the excess disinfection composition can be collected and distributed by gravity within an open or closed system, e.g., a gutter system. In another embodiment, the excess disinfection composition can be collected and stored for a suitable period of time before distribution.

In addition to applying the disinfection composition to a seafood product by spraying, the agent can be applied to the seafood product by dipping, brushing, electrostatic spray, and any other suitable means whereby a portion of the agent remains on the seafood product. In addition to removing the excess disinfection composition by gravity, the excess can additionally be removed by applying a centripetal force by rotating a seafood product, by suction, e.g., applying a vacuum to a seafood product, and any other suitable means. In each of the above embodiments, the excess disinfection composition can be used with the same additional disinfection composition and/or mixed with a different disinfection composition or combination of disinfection compositions. The pH and concentration of the solution applied to the seafood product can be adjusted by methods known to those of skill in the art. Such adjustments can also be accomplished by automated detection and titration systems known to those of skill in the art. In addition, filters and other clarifying apparatus can be provided at individual or multiple stations within the system or in the distribution of the disinfection composition. Furthermore, stations 2, 4 and, in the case of FIG. 2, station 6 can comprise additional sanitizing means, e.g., pressurized liquid sprayers, which can emit the same or different disinfection composition or a liquid that does not contain an disinfection composition.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Disinfection Composition Dip for Catfish

The effect of an acidic buffered copper containing disinfection composition was examined on shelf life, pathogen load, and odor for catfish fillets. The disinfection agent was commercially available Tasker Pacific Blue (including sulfuric acid, ammonium sulfate, copper sulfate, and water).

Treatment solution was prepared by filling a container with 2.0 liters of tap water, recording the initial pH, adding 0.13 ml of Tasker Pacific Blue concentrate (to provide mixture with target free copper concentration of 3 ppm), and manually mixing with a non-metal stir bar. After mixing, the resulting solution was measured for pH. Then, approximately 3.4 mL of Tasker Clear™ was titrated into the, allowing adequate time for mixing and equilibration, until a target pH of 2.0 was reached. The total amount of Tasker Clear™ is dependent upon the initial pH of the water.

Catfish fillets were secured from a catfish processor and dipped in the above 2.0 pH, 3 ppm copper Tasker Blue solution (or in water as controls) for 8, 15, or 20 min. The fillets were held at 2-4° C. for seven days. Fillets were then tested for psychrotrophic plate counts (PPC) and sensory odor (Kim and Silva, 2001) at days 1 and 7 of their refrigerated life.

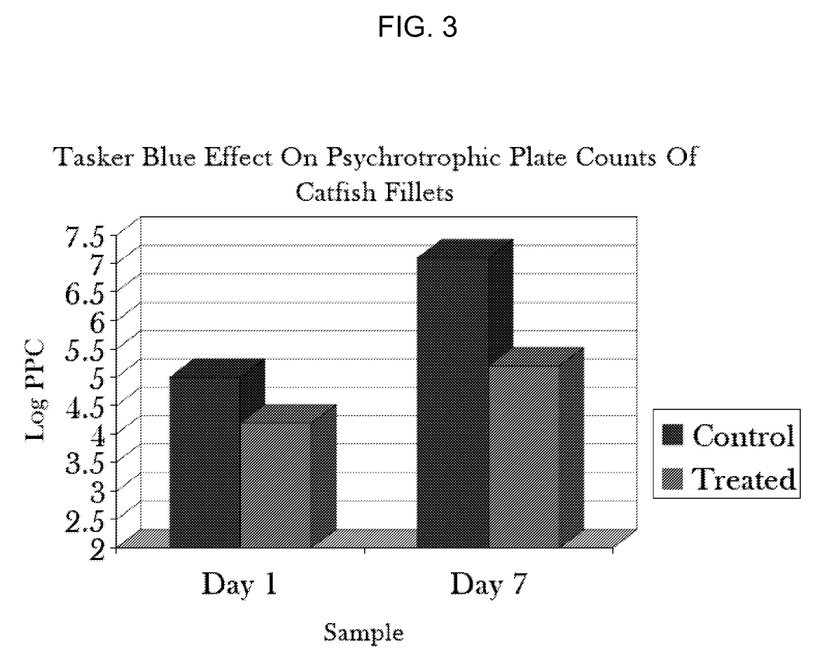

Results showed that fillets had an initial PPC of 5.0 (control) and 4.2-4.7 (treatments) (see e.g., Table 2; FIG. 3). After four days of storage, the control fillets produced a spoiled odor while the Tasker treated fillets were rated as “fresh”. On day 7, the control fillets had PPC of 7.1 (spoiled) while the Tasker treated fillets had PPCs of 5.2, 5.7, and 5.8 for contact times of 8, 15, and 20 minutes, respectively. Thus, Tasker Blue treated catfish fillets remained “fresh” on day 7 while control fillets are spoiled on day 4 of the tests (see e.g., Table 2; FIG. 3). Tasker Blue treatment effectively reduces PPC counts and extends shelf life by at least 3 days. And such results are accomplished with no negative organoleptic results. Further, effective results are accomplished by using a contact time of only 8 minutes.

These results demonstrate that the Tasker Blue solution can extend the lag phase and/or shift the microbial flora in catfish fillets such that it can accomplish up to double shelf life on refrigerated fillets.

TABLE 2
PPC count for catfish fillets treated with Tasker Blue

| | Time | | | |
|---|---|---|---|---|
| | Day 1 | | Day 7 | |
| | Control | Test | Control | Test |
| Dip Times | 8, 15, 20 | 8, 15, 20 | 8, 15, 20 | 8, 15, 20 |
| (Minutes) | | | | |
| Log PPC | 5 | 4.2-4.7 | 7.1 | 5.2, 5.7, 5.8 |
| Odor | | | Spoiled On Day 4 | Remained Fresh |
| | | | | On Day 7 |

### Example 2

### Disinfection Composition Dip for Catfish at a Commercial Processing Facility

The effect of an acidic buffered copper containing disinfection composition was examined on shelf life for catfish fillets at a large commercial catfish processing plant. The disinfection agent was commercially available Tasker Pacific Blue (including sulfuric acid, ammonium sulfate, copper sulfate, and water).

Catfish fillets were dipped in pH 2.3 and pH 3.0 solutions of Tasker Blue (3.0 ppm copper) at the rate of 0.88 lb fish per gallon of solution. The fillets were sampled after contact for up to 15 min. The experiment was then repeated with a second set of catfish fillets in the same solution. The pH was monitored after each dip group. The first set of fish in pH 2.3 was weighed, dipped, stirred several times, and drained. The second set of pre-chilled fillets were dipped in the same previous Tasker Blue solution and processed the same way as the first set. After the second set, pH of solution was 2.63. For pH 3 The pH of solutions, after treatment, dramatically increased to 4.8. Untreated chilled fillets were used as control

Fresh catfish fillets without chilling were treated with pH 2.2 or 3.0 Tasker Blue solution for 5, 10, and 15 min, separately. The initial pH 2.3 increased to 2.4, 2.4, and 2.5 after 5, 10, and 15 min, respectively. The initial 3.0 solution increased to 4.0, 4.0, and 4.8 after 5, 10, and 15 min, respectively. Microbial load (as total aerobic counts) was examined at days 1, 5, and 11 of storage at 4+/−1 C. Aerobic Plate Counts (APC) were determined using The Official Methods of Analysis of the AOAC, Method 990.12, and reported in colony forming units (CFU). E. coli were conducted using The Official Methods of Analysis of the AOAC, Method No. 990.12, and reported in colony forming units (CFU).

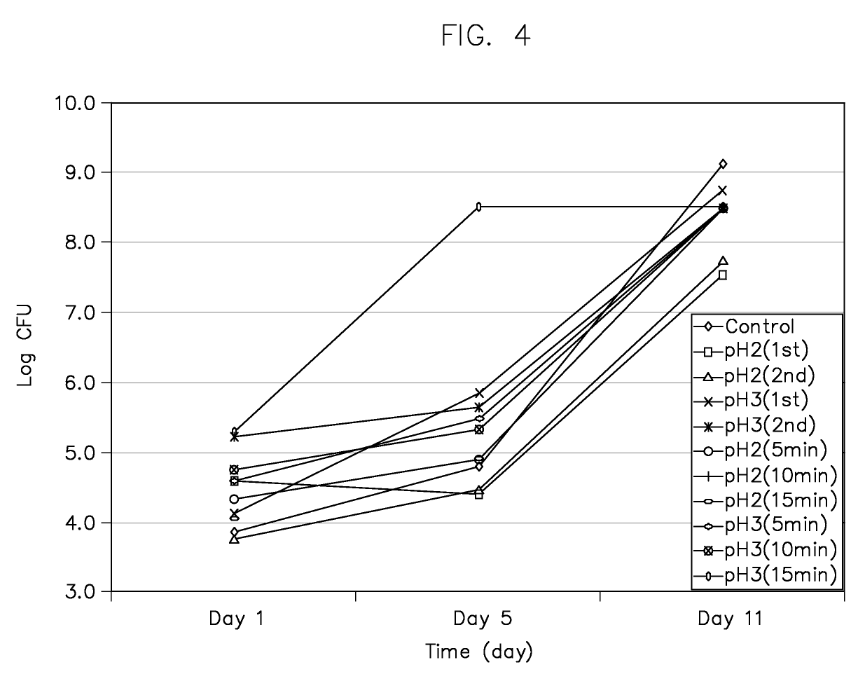

Results showed that, at day one, there was little difference in plate counts between treatment groups and control; but by day 11, fillets exposed to pH 2.3 solution had about 3 days longer shelf-life than control fillets (see e.g., FIG. 4). The pH 3 solution had less effect on plate count than the pH 2.3 solution. Contact time did not significantly influence shelf-life of fillets. These results suggest that fillets should be treated with Tasker Blue at pH near 2.0, and should be exposed to solution for short times.

Visual evaluation showed that treated fillets did not show signs of gapping or other adverse effects (e.g., protein denaturation) but were “whitened” by exposure to the solution. Such effect provides a positive market factor.

From this study, estimated consumption of Tasker Blue disinfectant was 7.55 gallons per day to treat 40,000 pounds of catfish fillets.

The results above confirm results that showed that Tasker Blue solution at pH 2 does not lower initial microbial load, but rather, changes the microflora and extends the lag phase of surviving organisms, increasing the shelf-life of the fillets. As such, Tasker Blue is an effective microbial intervention agent that can substantially reduce surface spoilage bacteria on fillets so as to contribute to enhanced shelf life.

### Example 3

### Disinfection Composition Dip for Salmon Fillets

The benefits of application of an acidic buffered copper containing disinfection composition in a dip application was evaluated as a means of reducing microbes and spoilage bacteria to extend shelf life of fresh salmon fillets. The disinfection agent was commercially available Tasker Pacific Blue (including sulfuric acid, ammonium sulfate, copper sulfate, and water).

Testing was performed at a commercial seafood processing plant. Seafood products tested were salmon fillets. Salmon fillets were weighed before and after treatment to determine absorption and consumption rates (see e.g., Table 3). Salmon fillets were dipped in the Tasker Pacific Blue Seafood Wash using a lugger tote (empty weight, 5.25 lbs, weight with Tasker Blue, 38.50 lbs). The pH levels of the solution were monitored with a hand held pH monitor between each filet treated. Each filet was randomly selected from a holding container. Salmon fillets were weighed and graded on the Roche color scale prior to dip treatment. Salmon fillets were then dipped and treated for 15 seconds or 30 seconds in treatment or control. Dipped salmon fillets were again weighed and graded on the Roche color scale. The Salmon fillets were then placed in a heavy duty shipping bags and held under refrigeration onsite for six days until testing. Salmon fillets were tested for aerobic plate count (APC), psychrotrophic plate count (PPC), and organoleptic odor/appearance (color scale when applicable).

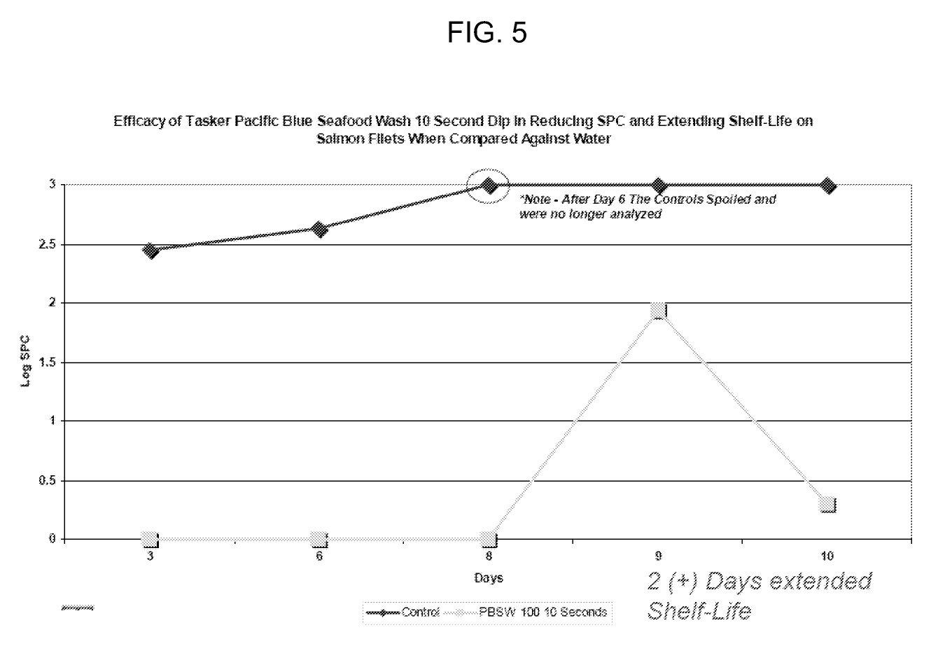

Results showed that Tasker Blue application to salmon fillets significantly reduced microbial growth on the fillets extended shelf-life by at least two days (see e.g., FIG. 5). Also, the initial pH of 2 did not change after treatment of all fillets.

TABLE 3

| Time | Initial Color | Color after dip | Initial weight | Weight after dip |
|---|---|---|---|---|
| 15 sec | 25 | 23 | 4.188 lbs | 4.266 lbs |
| 15 sec | 25 | 23 | 4.354 lbs | 4.409 lbs |
| 15 sec | 25 | 23 | 3.903 lbs | 3.986 lbs |
| 15 sec | 25 | 23 | 4.154 lbs | 4.241 lbs |
| 30 sec | 25 | 23 | 4.134 lbs | 4.185 lbs |
| 30 sec | 25 | 23 | 3.826 lbs | 3.882 lbs |
| 30 sec | 25 | 23 | 3.781 lbs | 3.825 lbs |
| 30 sec | 25 | 23 | 3.913 lbs | 3.957 lbs |

### Example 4

### Disinfection Composition Spray for Salmon Portions

The benefits of application of an acidic buffered copper containing disinfection composition in a spray application was evaluated as a means of reducing microbes and spoilage bacteria to extend shelf life of fresh salmon portions. The disinfection agent was commercially available Tasker Pacific Blue (including sulfuric acid, ammonium sulfate, copper sulfate, and water).

Testing was performed at a commercial seafood processing plant. Seafood products tested were salmon portions (6 oz). Each portion was randomly selected from a holding and graded on the Roche color scale. Salmon portions were sprayed with Tasker Pacific Blue Seafood Wash (pH 2.0, 3 ppm copper) for 10 seconds (150 ml), 15 seconds (200 ml), 30 seconds (350 ml), or 45 seconds (525 ml) (see e.g., Table 4). Products were sprayed on the top side only to mimic commercial process. Spray applications were applied with a hand held sprayer. After spraying, salmon portions were again graded on color. The treated salmon portions were then tray packed and held under refrigeration onsite for six days until testing. Salmon portions were tested for aerobic plate count (APC), psychrotrophic plate count (PPC), and organoleptic odor/appearance (color scale when applicable).

Results showed that Tasker Blue, at all exposure durations, lowered the levels of microbiological contamination on the salmon portions, extended the time during which the salmon portions had a fresh or neutral odor, and extended the time during which the salmon portions had an acceptable appearance, as compared to controls (see e.g., Table 6).

TABLE 4

| | Number of | | Color after | Total applied dosage |
|---|---|---|---|---|
| Test | portions | Initial Color | treatment | (volume) |
| Control | 7 | 23 | | |
| 10 sec | 7 | 23 | 23 | 150 ml PB100 |
| 15 sec | 7 | 23 | 23 | 200 ml PB100 |
| 30 sec | 7 | (2) @ 25 & | (2) @ 24 & | 350 ml PB100 |
| | | (5) @ 23 | (5) @ 23 | |
| 45 sec | 7 | 24 | 23 | 525 ml PB100 |

TABLE 5

| | Microbiological | | | Organoleptic | | | | | |
|---|---|---|---|---|---|---|---|---|---|
| Days (After | | Log | | | | | Odor | | |
| Test) | SPC | SPC | Coliform | Color | Texture | Odor | Score | Appearance | Temp |

Control

| | | | | | | | | | |
|---|---|---|---|---|---|---|---|---|---|
| 0 | 100 | 2 | 0.5 | 28 | 4 | fresh-melon | 0 | v. good | 38 |
| 3 | 280 | 2.447158 | 0.5 | 28 | 4 | fresh- | 0 | slight green on | 38 |
| | | | | | | protein | | fat | |
| 6 | 425 | 2.628389 | 0.5 | 28 | 4 | off | 2 | green fat | 38 |
| 8 | 1000 | 3 | | | | | | | |
| 9 | 1000 | 3 | | | | | | | |
| 10 | 1000 | 3 | | | | | | | |

Treated With O3

| | | | | | | | | | |
|---|---|---|---|---|---|---|---|---|---|
| 0 | | | | 28 | 4 | fresh-melon | 0 | v. good | 38 |
| 3 | 1 | 0 | 0.5 | 28 | 4 | fresh- | 0 | good | 38 |
| | | | | | | protein | | | |
| 6 | 1 | 0 | 0.5 | 28 | 4 | fresh | 0 | slight green on | 38 |
| | | | | | | | | fat | |
| 8 | 44 | 1.643453 | 0.5 | 28 | 4 | fresh | 0 | slight green on | 39 |
| | | | | | | | | fat | |
| 9 | 1 | 0 | 0.5 | 28 | 4 | neutral | 1 | green fat | 40 |
| 10 | 14 | 1.146128 | 0.5 | 28 | 4 | off | 2 | green fat | 42 |
| | | | | | | | | opaque flesh | |

Blue Dip 10 Seconds

| | | | | | | | | | |
|---|---|---|---|---|---|---|---|---|---|
| 3 | 1 | 0 | 0.5 | 30 | 4 | Fresh | 0 | v. good | 39 |
| 6 | 1 | 0 | 0.5 | 28 | 4 | Fresh | 0 | good | 38 |
| 8 | 1 | 0 | 0.5 | 28 | 4 | Fresh | 0 | slight green on | 39 |
| | | | | | | | | fat | |
| 9 | 87 | 1.939519 | 0.5 | 28 | 4 | Neutral | 1 | slight green on | 40 |
| | | | | | | | | fat | |
| 10 | 2 | 0.30103 | 0.5 | 26 | 4 | Off | 2 | green fat | 42 |
| | | | | | | | | opaque flesh | |

Blue Dip 15 Seconds

| | | | | | | | | | |
|---|---|---|---|---|---|---|---|---|---|
| 3 | 1 | 0 | 0.5 | 30 | 4 | Fresh | 0 | v. good | 39 |
| 6 | 2 | 0.30103 | 0.5 | 28 | 4 | Fresh | 0 | good | 38 |
| 8 | 4 | 0.60206 | 0.5 | 28 | 4 | Fresh | 0 | slight green on | 39 |
| | | | | | | | | fat | |
| 9 | 100 | 2 | 0.5 | 26 | 4 | Neutral | 1 | slight green on | 39 |
| | | | | | | | | fat | |
| 10 | 1 | 0 | 0.5 | 26 | 4 | Off | 2 | green fat | 42 |
| | | | | | | | | opaque flesh | |

Blue Dip 30 Seconds

| | | | | | | | | | |
|---|---|---|---|---|---|---|---|---|---|
| 3 | 1 | 0 | 0.5 | 28 | 4 | Fresh | 0 | v. good | 38 |
| 6 | 1 | 0 | 0.5 | 28 | 4 | Fresh | 0 | good | 38 |
| 8 | 16 | 1.20412 | 0.5 | 28 | 4 | Fresh | 0 | slight green on | 39 |
| | | | | | | | | fat | |
| 9 | 305 | 2.4843 | 0.5 | 28 | 4 | Neutral | 1 | slight green on | 41 |
| | | | | | | | | fat | |
| 10 | 280 | 2.447158 | 12 | 26 | 4 | Off | 2 | green fat | 42 |

Blue Dip 45 Seconds

| | | | | | | | | | |
|---|---|---|---|---|---|---|---|---|---|
| 3 | 1 | 0 | 0.5 | 28 | 4 | Fresh | 0 | v. good | 38 |
| 6 | 1 | 0 | 0.5 | 28 | 4 | Fresh | 0 | good | 38 |
| 8 | 41 | 1.612784 | 0.5 | 28 | 4 | Fresh | 0 | slight green on | 40 |
| | | | | | | | | fat | |
| 9 | 200 | 2.30103 | 0.5 | 28 | 4 | Neutral | 1 | slight green on | 40 |
| | | | | | | | | fat | |
| 10 | 6 | 0.778151 | 5 | 26 | 4 | Off | 2 | green fat | 42 |

### Example 5

### Disinfection Composition Dip for Shrimp and Scallops

The effect of an acidic buffered copper containing disinfection composition applied as a dip was examined on pathogen load, organoleptic characteristics, and odor for shrimp and scallops. The disinfection agent was commercially available Tasker Pacific Blue (including sulfuric acid, ammonium sulfate, copper sulfate, and water).

The Tasker Pacific Blue Seafood Wash solution (pH 2, 3 ppm copper) was prepared in 2 liters water. Fresh raw shrimp and fresh raw scallops dipped for 1 minute, then divided into bags. The samples were held at 40° F. and 45° F. for shelf-life analysis. Samples were tested for APC, E. coli, organoleptic characteristics, and odor at days 2, 5, 8, 12, & 16.

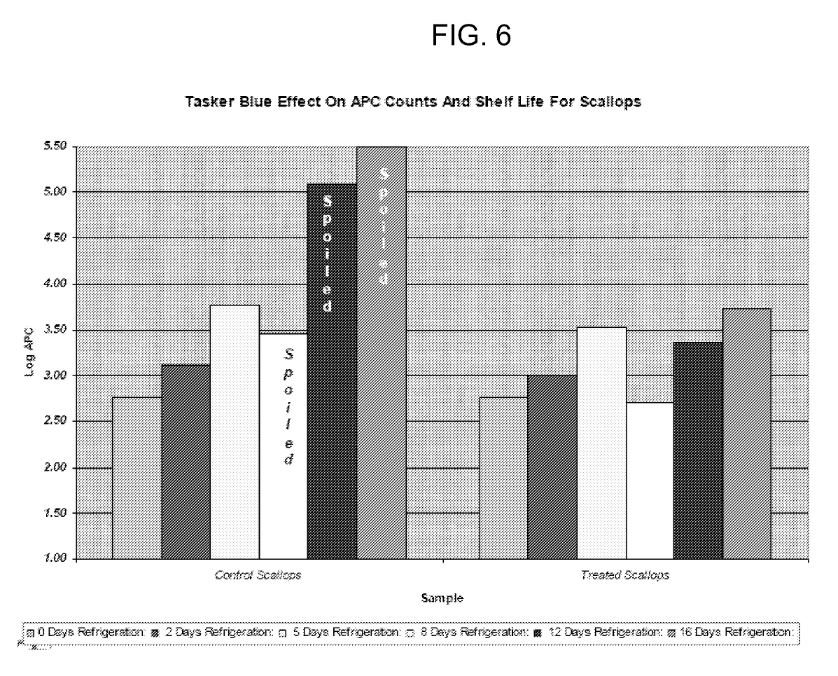

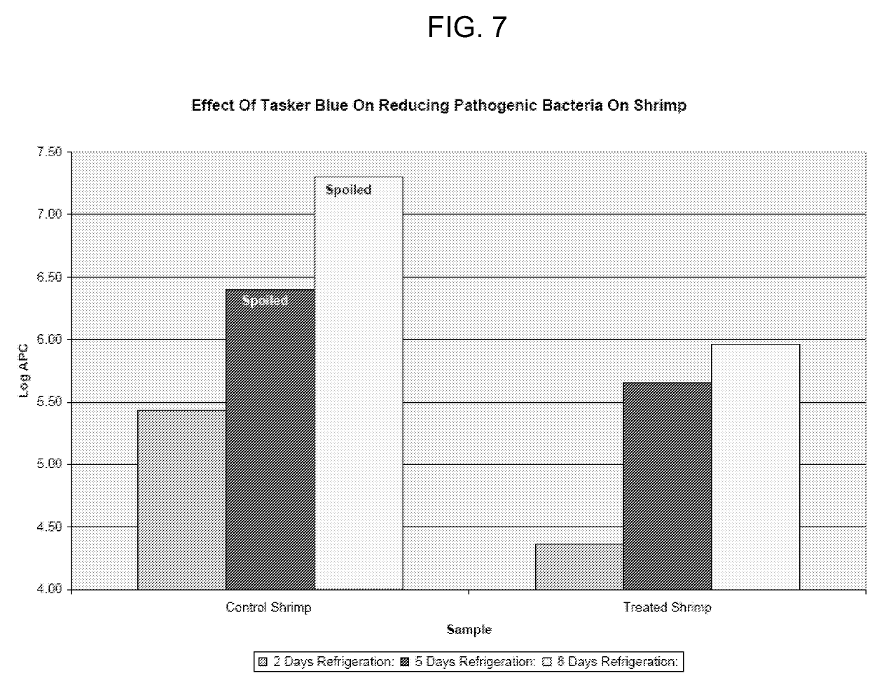

Results showed that Tasker Blue dramatically reduced E. coli and coliform bacteria and increased shelf life for both shrimp and scallops (see e.g., FIG. 6; FIG. 7; Table 6). For scallops, control samples spoiled on day 8 while treated samples remained “fresh” on day 16. Tasker Blue treatment reduced APC in scallops by approximately 1 log on day 12. For shrimp, After 8 days of refrigeration the treated shrimp had approximately a 2 log reduction in APC. Tasker Blue Treatment had fresh odor at day 8 while control odor spoils on day 5.

TABLE 6
0 Days Refrigeration:

| Sample | Initial APC | T. Coliform | E. coli | |
|---|---|---|---|---|
| Shrimp | 100,000 | >1000 | <10 | 5.00 |
| Scallops | 590 | <10 | <10 | 2.77 |

2 Days Refrigeration:

| Sample | Temp. ° F. | APC | T. Coliform | E. coli | Log APC | |
|---|---|---|---|---|---|---|
| Control Shrimp | 40 | 270,000 | 15,000 | 600 | 5.43 | Sensory indicates |
| Treated Shrimp | 40 | 23,000 | 700 | <100 | 4.36 | treatment masks |
| Control Shrimp | 45 | 160,000 | 20,000 | 300 | 5.20 | odor in shrimp and |
| Treated Shrimp | 45 | 60,000 | 2,000 | 30 | 4.78 | Scallops |
| Control Scallops | 40 | 1,300 | <10 | <10 | 3.11 | |
| Treated Scallops | 40 | 1,000 | <10 | <10 | 3.00 | |
| Control Scallops | 45 | 1,700 | <10 | <10 | 3.23 | |
| Treated Scallops | 45 | 2,000 | <10 | <10 | 3.30 | |

5 Days Refrigeration:

| Sample | Temp. ° F. | APC | T. Coliform | E. coli | | |
|---|---|---|---|---|---|---|
| Control Shrimp | 40 | 2,500,000 | 44,000 | 5,000 | 6.40 | Shrimp smell spoiled |
| Treated Shrimp | 40 | 450,000 | 450 | 250 | 5.65 | |
| Control Shrimp | 45 | 560,000 | 16,000 | 1,000 | 5.75 | |
| Treated Shrimp | 45 | 1,200,000 | 10,000 | 3,600 | 6.08 | |
| Control Scallops | 40 | 5,800 | <10 | <10 | 3.76 | Scallop odor still |
| Treated Scallops | 40 | 3,400 | <10 | <10 | 3.53 | fresh in treated |
| Control Scallops | 45 | 3,600 | 10 | <10 | 3.56 | samples |
| Treated Scallops | 45 | 2,000 | <10 | <10 | 3.30 | |

8 Days Refrigeration:

| Sample | Temp. ° F. | APC | T. Coliform | E. coli | | |
|---|---|---|---|---|---|---|
| Control Shrimp | 40 | 20,000,000 | 12,000 | 5,000 | 7.30 | |
| Treated Shrimp | 40 | 920,000 | 640 | 570 | 5.96 | |
| Control Shrimp | 45 | 5,800,000 | 11,000 | 2,000 | 6.76 | |
| Treated Shrimp | 45 | 3,500,000 | 20,000 | 7,000 | 6.54 | |
| Control Scallops | 40 | 2,800 | <10 | <10 | 3.45 | Sensory indicates |
| Treated Scallops | 40 | 500 | <10 | <10 | 2.70 | spoiled product |
| Control Scallops | 45 | 5,000 | <10 | <10 | 3.70 | on all control |
| | | | | | | Scallops |
| Treated Scallops | 45 | 4,500 | <10 | <10 | 3.65 | |

12 Days Refrigeration:

| Sample | Temp. ° F. | APC | Yeast/Mold | |
|---|---|---|---|---|
| Control Shrimp | 40 | discarded | | |
| Treated Shrimp | 40 | discarded | | |
| Control Shrimp | 45 | discarded | | |
| Treated Shrimp | 45 | discarded | | |
| Control Scallops | 40 | 125,000 | 8,600 | 5.10 |
| Treated Scallops | 40 | 2,300 | 300 | 3.36 |
| Control Scallops | 45 | 1,000 | <100 | 3.00 |
| Treated Scallops | 45 | 2,300 | 400 | 3.36 |

16 Days Refrigeration:

| Sample | Temp. ° F. | APC | Yeast/Mold | | |
|---|---|---|---|---|---|
| Treated Scallops | 40 | 5,400 | <10 | 3.73 | Sensory indicates |
| Treated Scallops | 40 | 1,300 | <10 | 3.11 | fresh smell |
| Treated Scallops | 40 | 1,800 | <10 | 3.26 | maintaining |
| Treated Scallops | 45 | 21,000 | <10 | 4.32 | |
| Treated Scallops | 45 | 30,000 | <10 | 4.48 | |
| Treated Scallops | 45 | 18,000 | <10 | 4.26 | |

### Example 6

### Disinfection Composition Ice in Seafood Cabinet

The effect of an acidic buffered copper containing disinfection composition in ice was examined for reducing odors on fish fillets. The disinfection agent was commercially available Tasker Pacific Blue (including sulfuric acid, ammonium sulfate, copper sulfate, and water).

Ice was made with the following substances added to the water prior to the ice-making: (1) Control (only tap water); (2) 5 ppm Ozone; (3) 100 ppm Peracetic acid; or (4) Tasker Blue (PBSW-100).

Raw channel catfish fillets were obtained from a catfish plant and placed on the different ice treatments in a refrigerator at 2-4° C. for 14 days to simulate a seafood counter in a retail store. The ice was made daily and changed accordingly so that the fish would always be on ice. Scores for odor (off-odor as spoilage, putrid, or other) were started when the sensory panelists felt there were differences (day 11). Prior to this day, little effect of treatment on odor was observed. The panelists scored the odor on a 9-point scale with 9 (extremely pleasant, fresh) to 1 (extremely putrid, foul smell).

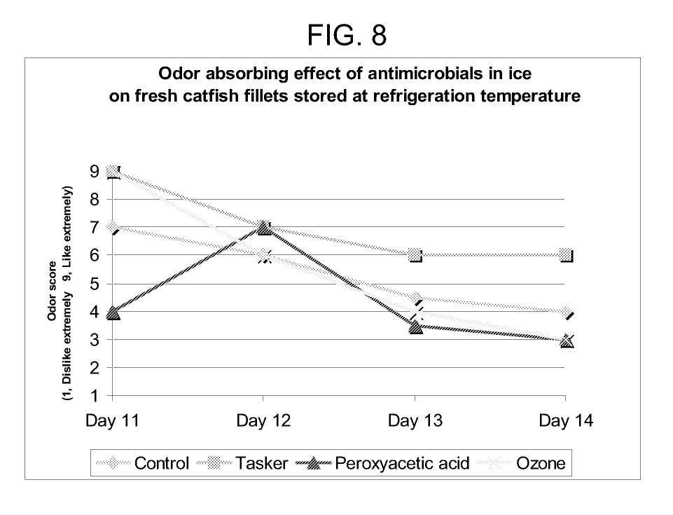

Results showed that the ozonated ice and the Tasker Blue ice were both considered good on day 11 but the fish on ozonated ice started to decrease in odor score preference after day 12 (see e.g., FIG. 8). This is possibly due to the short lifespan of ozone. The peracetic acid treatment itself produced an off-odor smell itself even from day 11. On day 14, the fish on the Tasker Blue ice were still acceptable (odor less than strong) as compared to the other treatments and the control. This experiment produced similar as an earlier preliminary experiment.

These results suggest that ice containing Tasker Blue is able to absorb foul odors from fresh fish, increasing time to spoilage and thus increasing display time. This approach can also benefit storage of other seafood products. Further, Tasker Blue containing ice can reduce the cross-contamination that occurs from nightly transfer of seafood product from the display case to overnight freezer/cooler storage.

### Example 7

### Organoleptic and Odor Tests

The effects of an acidic buffered copper containing disinfection composition on organoleptic characteristics and odor was examined for mahi-mahi fillets, skinless flounder fillets, and shrimp. The disinfection agent was commercially available Tasker Pacific Blue (including sulfuric acid, ammonium sulfate, copper sulfate, and water).

Mahi-Mahi fillets were dipped into a Tasker Pacific Blue Seafood solution and samples placed in permeable bags and stored on ice for 6 days. Skinless flounder fillets and (head-off) shrimp were dipped into the Tasker Pacific Blue Seafood solution and samples placed in permeable bags and stored on ice until the next day.

Results showed that Tasker Blue dip was very effective at reducing odor in mahi-mahi fillets, skinless flounder fillets, and shrimp. Tasker Blue did not cause any noticeable color change, flavor change, or oxidation of shrimp. There was observed a slight darkening of treated mahi-mahi fillets and a slight, but not objectionable, difference was noted on cooked flounder fillets.

### Example 8

### Seafood Sensory Testing

The effect of an acidic buffered copper containing disinfection composition was examined on sensory characteristics for shrimp, croaker, salmon filet, whole red snapper (dipped and iced). The disinfection agent was commercially available Tasker Pacific Blue (including sulfuric acid, ammonium sulfate, copper sulfate, and water).

Tasker Blue treatment solutions included pH of 2 or 3.5 and copper content of 1 ppm, 2 ppm, or 3 ppm. Shrimp and Croaker were sprayed evenly with the Tasker Blue solution. Salmon filet and whole red Snapper were dipped in the Tasker Blue solution for 5 minutes (croaker), 30 seconds (salmon filet), or 1 and 5 minutes (red Snapper). Another group of whole red snapper was also stored on ice made with Tasker Blue for 12 hours at 40 F. Odor was ranked on a scale of 1-10, where 10 was heavy, 7 was moderate, 4 was slight, and 1 was no odor.

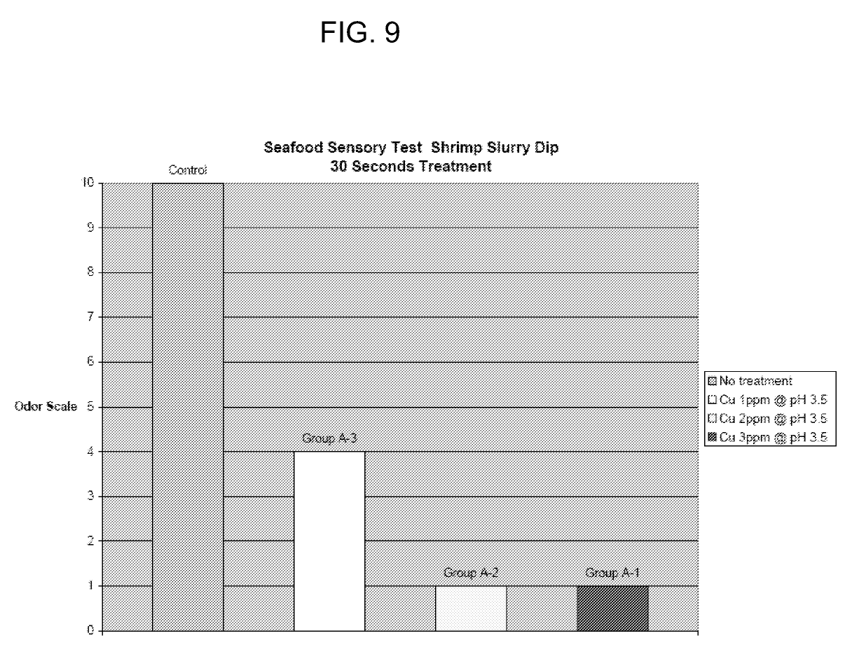

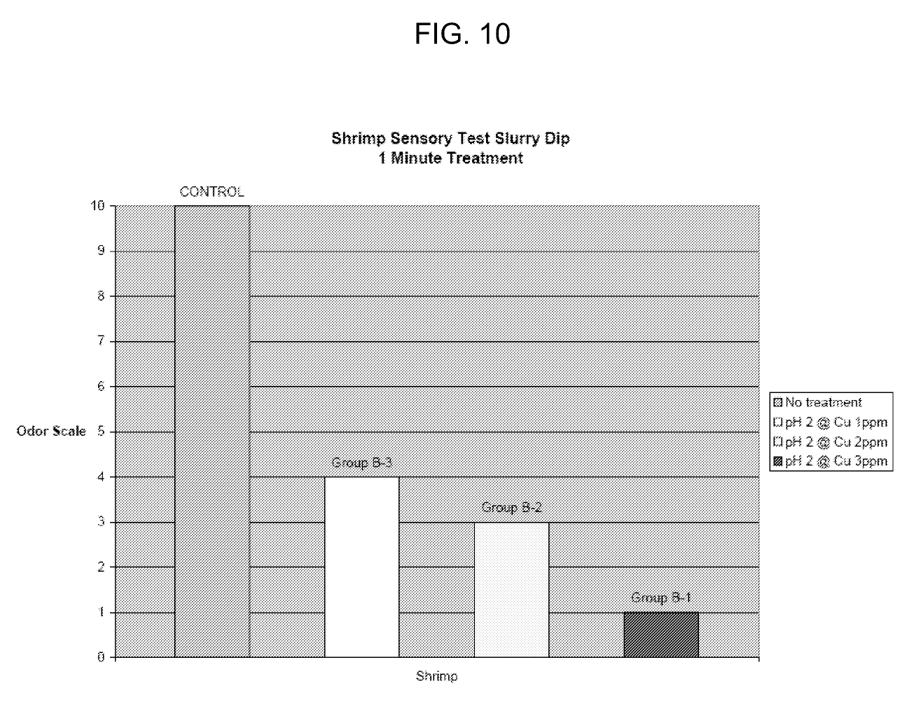

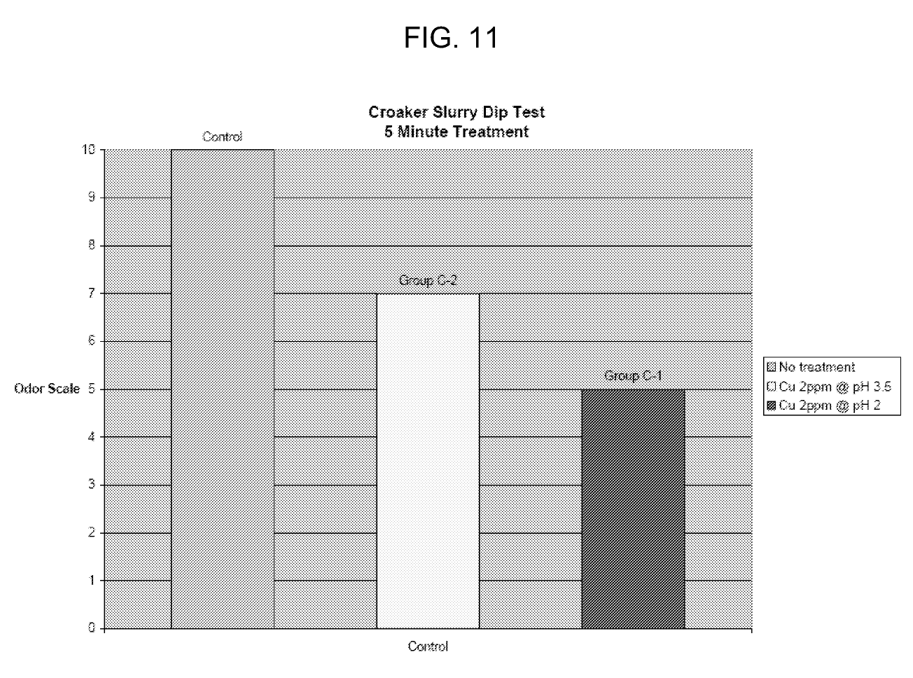

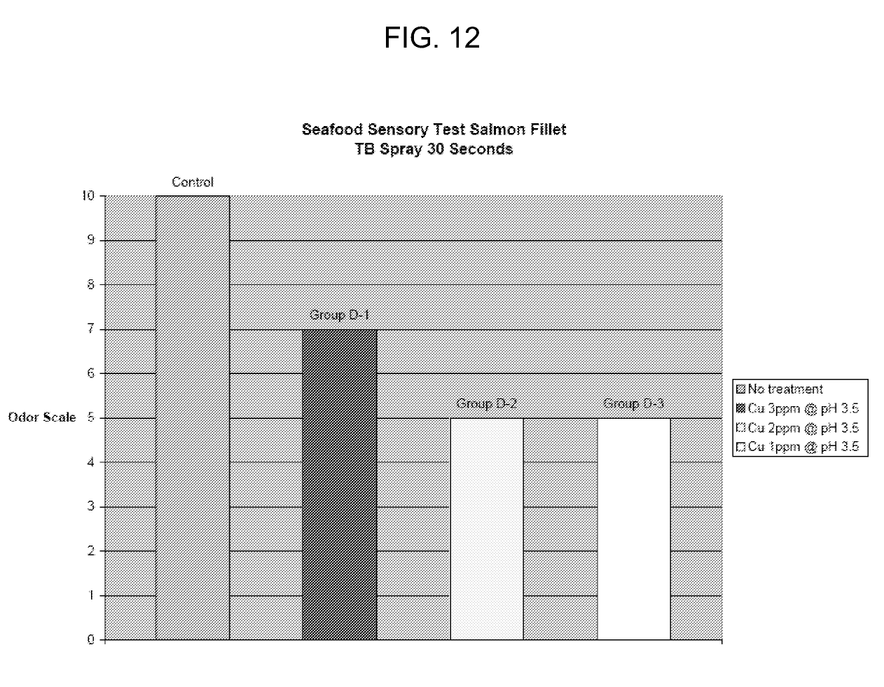

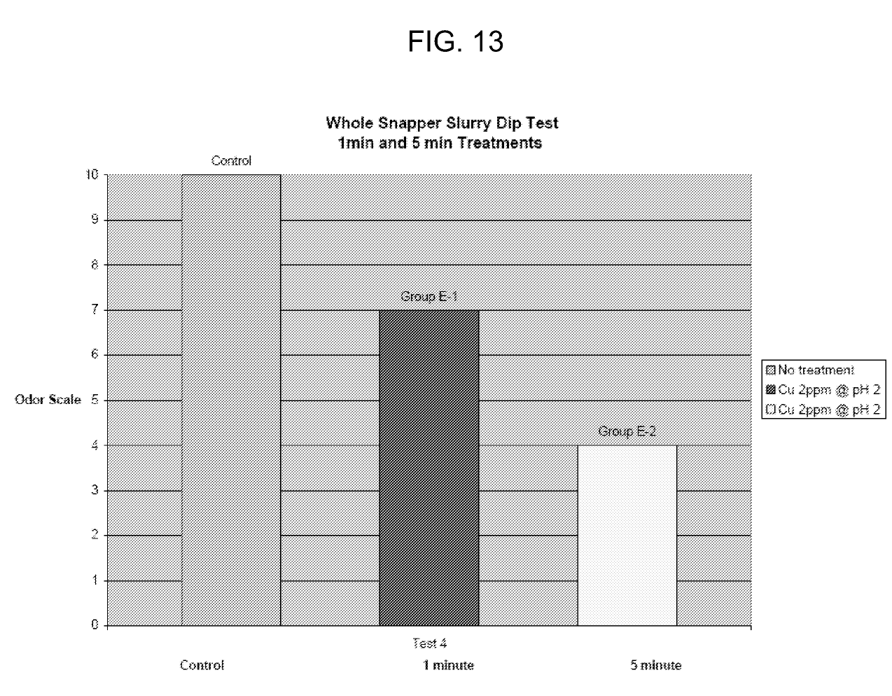

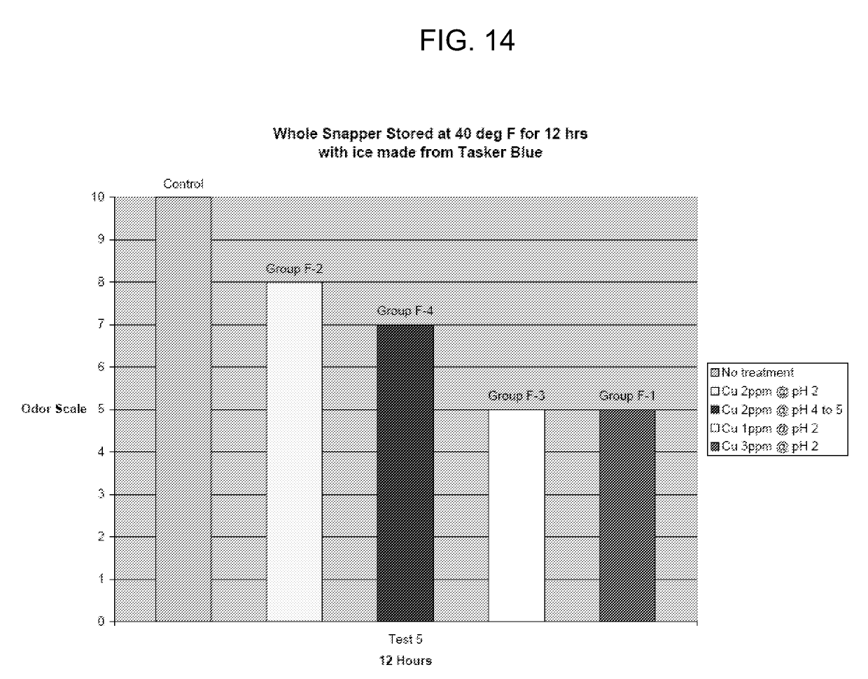

Results showed that Tasker Blue dip was effective at reducing odor in all treatments for shrimp, croaker, salmon filet, and whole snapper (see e.g., Table 7; FIG. 9; FIG. 10; FIG. 11; FIG. 12; FIG. 13). Also, Tasker Blue ice was effective at reducing odor on whole snapper in all treatments (see e.g., FIG. 14).

TABLE 7
Effect of Tasker Blue on odor for shrimp, croaker, salmon
fillets, and whole snapper. Odor is reported 1-10, where 10 is heavy, 7
is moderate, 4 is slight, and 1 is no odor.

| | | |
|---|---|---|
| Tasker Blue Dosage | Time | Results |

Seafood Sensory Testing Shrimp Dip

| | | | |
|---|---|---|---|
| Test 1 B | | | |
| Control | No treatment | | 10 |
| Group A-1 | Cu 3 ppm @ pH 3.5 | 30 sec | 1 |
| Group A-2 | Cu 2 ppm @ pH 3.5 | 30 sec | 1 |
| Group A-3 | Cu 1 ppm @ pH 3.5 | 30 sec | 4 |
| Group A-4 | pH 3.5 @ Cu 2 ppm | 10 min | 1 |
| Control | No treatment | | 10 |
| Group B-1 | pH 2 @ Cu 3 ppm | 1 min | 1 |
| Group B-2 | pH 2 @ Cu 2 ppm | 1 min | 3 |
| Group B-3 | pH 2 @ Cu 1 ppm | 1 min | 4 |

Seafood Sensory Testing Whole Croaker Slurry Dip

| | | | |
|---|---|---|---|
| Test 2 | | | |
| Control | No treatment | | 10 |
| Group C-1 | Cu 2 ppm @ pH 2 | 5 min | 5 |
| Group C-2 | Cu 2 ppm @ pH 3.5 | 5 min | 7 |

Seafood Sensory Testing Salmon Fillet TB Spray

| | | | |
|---|---|---|---|
| Test 3 | | | |
| Control | No treatment | | 10 |
| Group D-1 | Cu 3 ppm @ pH 3.5 | 30 sec | 7 |
| Group D-2 | Cu 2 ppm @ pH 3.5 | 30 sec | 5 |
| Group D-3 | Cu 1 ppm @ pH 3.5 | 30 sec | 5 |

Seafood Sensory Testing Whole Red Snapper Slurry Dip

| | | | |
|---|---|---|---|
| Test 4 | | | |
| Control | No treatment | | 10 |
| Group E-1 | Cu 2 ppm @ pH 2 | 1 min | 7 |
| Group E-2 | Cu 2 ppm @ pH 2 | 5 min | 4 |

Seafood Sensory Testing Whole Snapper stored w/ice made with TB

| Test 5 | Tasker Blue Dosage | Time | Temperature | Results |
|---|---|---|---|---|
| Control | No treatment | 12 hrs | 40 F | 10 |
| Group F-1 | Cu 3 ppm @ pH 2 | 12 hrs | 40 F. | 5 |
| Group F-2 | Cu 2 ppm @ pH 2 | 12 hrs | 40 F. | 8 |
| Group F-3 | Cu 1 ppm @ pH 2 | 12 hrs | 40 F. | 5 |
| Group F-4 | Cu 2 ppm @ pH 4 to 5 | 12 hrs | 40 F. | 7 |

### Example 9

### Disinfection Composition Effect on Listeria monocytogenes

The effect of an acidic buffered copper containing disinfection composition was examined on Listeria monocytogenes. The disinfection agent was commercially available Tasker Pacific Blue (including sulfuric acid, ammonium sulfate, copper sulfate, and water).

Twelve tubes of nutrient broth were inoculated with 10 ul of culture (L. mono., Scott A strain, log phase growth at 10⁸ cfu/ml) to contain 10⁵ cfu/ml Listeria monocytogenes. 1 ml of peptone buffer is added to the control samples (6). 1 ml of Tasker 10× solution (pH 2.8, 3 ppm copper) to treated samples (6). Tubes stored at 40° F. for 4 days and then analyzed for Listeria monocytogenes.

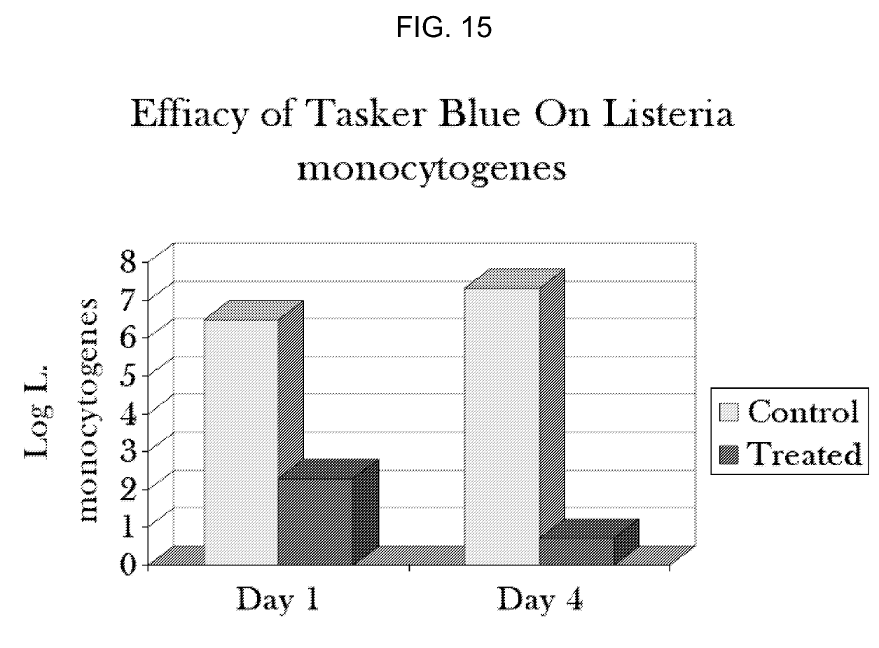

Results showed that controls had 3,000,000 cfu/ml at Day 1 and 21,000,000 cfu/ml at Day 7. Treated groups had 200 cfu/ml at Day 1 and <10 cfu/ml at Day 7 (see e.g., FIG. 15; Table 8).

TABLE 8
Tasker Blue effect on L. Mono content
After 24 hours, plated on Palcam Agar:

| | | | |
|---|---|---|---|
| 40° F. | control | 3,000,000 | 6.477121 |
| | treated | 200 | 2.30103 |
| 45° F. | control | 3,000,000 | 6.477121 |
| | treated | 1,100 | 3.041393 |

After 4 Days:

| | | | | |
|---|---|---|---|---|
| 40° F. | control | 21,000,000 | 7.322219 | |
| | treated | 10 | 1 | 0.7 |
| 45° F. | control | 43,000,000 cfu/ml | | |
| | treated | <10 cfu/ml | | |

### Example 10

### Disinfection Composition Effect on E. coli

The inhibitory activity of acidic buffered copper-containing disinfection agents was determined against Escherichia coli ATCC 11229. The disinfection agent was commercially available Tasker Blue (including sulfuric acid, ammonium sulfate, copper sulfate pentahydrate, and water).

Test samples were prepared for testing at pH levels of 2.0, 2.5, 3.0, 3.5, and 4.0 in combination with copper concentrations of 0 ppm, 1 ppm, 2 ppm, and 3 ppm. Tryptic soy Broth was prepared half strength as a standard inoculum of 0.5 McFarland. The test sample was added to a sterile tube, along with the same amount of standardized Escherichia coli ATCC 11229 inoculum. The pH of the sample was recorded and adjusted as indicated on the test sample bottle. Tubes were incubated for 24 hours at 35° C. and the inhibitory concentration was determined as the lowest concentration showing visible inhibition of the growth of the organism. All samples were run in duplicate along with positive and negative growth controls. Final pH of test samples were recorded following completion of 24 hour incubation.

Results showed that complete inhibition of microbial growth was achieved with all solutions except the following solutions, in which microbial growth was detected: pH 4.0 Cu 0 ppm; pH 4.0 Cu 1 ppm; pH 4.0 Cu 2 ppm; pH 4.0 Cu 3 ppm.

### Example 11

### Disinfectant Formulations

The inhibitory activity of acidic buffered disinfection agents on aerobic plate count (APC) was examined. Five formulations were tested.

Mark I: a 24 hour high temperature reaction process at approximately 300-350° F. with a stabilization step after overnight cooling. Composed of reacting 98% sulfuric acid with a 26-28% by weight ammonium sulfate in water solution. The order of addition was ammonium sulfate solution to sulfuric acid. Electrolysis of the reacting solution was applied for 1 hour at the start of the process. The stabilization step was the addition of more ammonium sulfate solution to ensure that the reaction is complete. The Tasker Clear™ product formed was a buffered acid solution of a strong acid (sulfuric acid) and a salt (ammonium sulfate) of a strong acid and strong base.

Mark II: a 2 hour low temperature reaction process at approximately 200-210° F. with a stabilization step immediately after the 1 hour electrolysis period. This was the same process as in the Mark I product above except that it was performed at a lower temperature and a shorter period of time. The ingredient amounts were adjusted to account for no lost of water as was seen in the Mark I process. The Tasker Clear™ product formed was a buffered acid solution of a strong acid (sulfuric acid) and a salt (ammonium sulfate) of a strong acid and strong base.

Mark III: a low temperature reaction process in which the 98% sulfuric acid was added slowly to a 30% by weight ammonium sulfate solution. The addition was done continuously until all the ammonium sulfate solution was added. There was no stabilization step. The addition order was the reverse of the Mark I, II, IV, and V processes. The temperature was maintained in the 150-200° F. range during the addition process. No electrolysis was performed during this process and hence the name ‘cold process’ was given to it. The Tasker Clear™ product formed was a buffered acid solution of a strong acid (sulfuric acid) and a salt (ammonium sulfate) of a strong acid and strong base.

Mark IV: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. Electrolysis of the reacting solution was applied for 1 hour at the start of the process. The stabilization step was the addition of more sodium sulfate solution to ensure that the reaction is complete. The Tasker Clear™ product formed was a buffered acid solution of a strong acid (sulfuric acid) and a salt (sodium sulfate) of a strong acid and strong base. (Note: In this process sodium sulfate was substituted for ammonium sulfate.)

Mark V: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. There was no electrolysis during this process (cold process). The stabilization step was the addition of more sodium sulfate solution to ensure that the reaction was complete. The Tasker Clear™ product formed was a buffered acid solution of a strong acid (sulfuric acid) and a salt (sodium sulfate) of a strong acid and strong base. (Note: In this process sodium sulfate was substituted for ammonium sulfate, and no electrolysis was performed.)

Results showed that all formulations exponentially reduced the aerobic plate count (see e.g., Table 9).

TABLE 9
Butterfield Buffer Control

| | Counts | Log₁₀ |
|---|---|---|
| Time | cfu/ml | cfu/ml |
| 0 | 845 | 2.93 |
| 5 | 780 | 2.89 |
| 15 | 785 | 2.89 |
| | | Ave = 2.90 |

DI Water Control

| Time | Counts cfu/ml | Log₁₀ cfu/ml |
|---|---|---|
| 0 | 1015 | 3.01 |
| 5 | 1075 | 3.03 |
| 15 | 940 | 2.97 |
| | | Ave = 3.00 |

Mark I Solution

| | Counts | Log₁₀ | Log |
|---|---|---|---|
| Time | cfu/ml | cfu/ml | Reduction |
| 0 | 140 | 2.15 | 0.85 |
| 5 | 25 | 1.40 | 1.60 |
| 15 | 5 | 0.70 | 2.30 |

Mark II Solution

| | | | Log |
|---|---|---|---|
| Time | Counts cfu/ml | Log₁₀ cfu/ml | Reduction |
| 0 | 100 | 2.00 | 1.00 |
| 5 | 30 | 1.48 | 1.52 |
| 15 | 0 | 0.00 | 3.00 |

Mark III Solution

| | Counts | Log₁₀ | Log |
|---|---|---|---|
| Time | cfu/ml | cfu/ml | Reduction |
| 0 | 65 | 1.81 | 1.19 |
| 5 | 0 | 0.00 | 3.00 |
| 15 | 0 | 0.00 | 3.00 |

Mark IV Solution

| | | | Log |
|---|---|---|---|
| Time | Counts cfu/ml | Log₁₀ cfu/ml | Reduction |
| 0 | 110 | 2.04 | 0.96 |
| 5 | 40 | 1.60 | 1.40 |
| 15 | 0 | 0.00 | 3.00 |

Mark V Solution

| | Counts | Log₁₀ | Log |
|---|---|---|---|
| Time | cfu/ml | cfu/ml | Reduction |
| 0 | 125 | 2.10 | 0.90 |
| 5 | 20 | 1.30 | 1.70 |
| 15 | 5 | 0.70 | 2.30 |

NOTES:
* Log Reduction based on DI Water average log₁₀ = 3.00
** Counts are the average of duplicate APC plates

What is claimed is:

1. A method of reducing a microbial population on seafood during processing comprising the steps of contacting a seafood during processing with a disinfection composition comprising an acid, a buffer, and an antimicrobial metal in an amount and time sufficient to reduce a microbial population.

2. The method of claim 1 comprising:
(a) contacting a seafood during processing with a disinfection composition comprising an acid, a buffer, and an antimicrobial metal in an amount and time sufficient to reduce a microbial population;
and further comprising at least one step selected from the group consisting of:
(b) harvesting the seafood;
(c) storing the seafood;
(d) stunning the seafood;
(e) deheading the seafood;
(f) eviscerating the seafood;
(g) skinning the seafood;
(h) chilling the seafood;
(i) trimming the seafood;
(j) washing the seafood;
(k) glazing the seafood before freezing;
(l) packaging the seafood;
(m) transporting the seafood; and
(n) displaying the seafood in a commercial venue;
wherein contacting the seafood with the disinfection composition (a) is performed during or immediately after at least one of steps b, c, d, e, f, g, h, i, j, k, l, m, or n.

3. The method of claim 2 further comprising the steps of:
(o) recovering at least a portion of the disinfection composition contacted with the seafood;
(p) adding to the recovered composition a sufficient amount of disinfection composition to yield a recycled disinfection composition; and
(q) contacting the seafood with the recycled disinfection composition during processing;
wherein contacting the seafood with the recycled disinfection composition (q) is performed during or immediately after at least one of steps b, c, d, e, f, g, h, i, j, k, l, m, or n.

4. The method of claim 1 wherein contacting the seafood with the disinfection composition comprises submersing or spraying the seafood in or with the disinfection composition.

5. The method of claim 2 wherein contacting the seafood with the disinfection composition is performed at least during holding the seafood and chilling the seafood and immediately after trimming the seafood.

6. The method claim 5 wherein contacting the seafood with the disinfection composition is performed at least during holding the seafood, chilling the seafood, and glazing the seafood and immediately after trimming the seafood.

7. The method claim 5 wherein contacting the disinfection composition with the seafood immediately after trimming the seafood is performed at a sanitization station, wherein the sanitization station is intermittently fluidly connected to an apparatus for holding; an apparatus for chilling, an apparatus for skinning; or to a combination thereof, so as to allow transfer of recycled disinfection composition from the sanitization station to the holding apparatus, the chilling apparatus, the skinning apparatus, or to a combination thereof.

8. The method of claim 1 wherein contacting the seafood with the disinfection agent reduces odor of the seafood; delays onset of odor of the seafood; extends shelf-life of the seafood; does not significantly depreciate the organoleptic properties of the seafood; or a combination thereof.

9. The method of claim 1 wherein contacting the seafood with the disinfection composition inhibits glycolytic, proteolytic, or lipolytic enzymatic seafood degradation, or a combination thereof.

10. The method of claim 1 wherein the seafood comprises a fish, a crustacean, or a mollusc.

11. The method of claim 11 wherein the seafood is a fish.

12. The method of claim 11 wherein the seafood comprises a fish selected from the list consisting of anchovy, barramundi, bass, butterfish, carp, catfish, capelin, cod, croaker, eel, flounder, flathead, flatfish, groundfish, haddock, halibut, harvestfish, hilsa, herring, John Dory, kapenta, mackerel, mahi-mahi, milkfish, monkfish, orange roughy, saury, panfish, pollock, pilchard, redfish, salmon, sardine, scrod, sea bass, seer fish, shad, shrimpfish, silver carp, skate, snapper, snook, snoek, sole, sturgeon, swordfish, tilapia, trout, tuna, turbot, walleye, walu, whitebait, whitefish, and whiting.

13. The method of claim 1 wherein the disinfection composition has a pH of about 1.5 to about 6.

14. The method of claim 13 wherein the disinfection composition has a pH of about 1.5 to about 4.

15. The method of claim 14 wherein the disinfection composition has a pH of about 2 to about 3.

16. The method of claim 15 wherein the disinfection composition has a pH of about 2.

17. The method of claim 1 wherein the disinfection composition comprises (i) sulfuric acid and (ii) ammonium sulfate or sodium sulfate.

18. The method of claim 1 wherein the antimicrobial metal is copper, zinc, magnesium, or silver.

19. The method of claim 18 wherein the disinfection composition has a copper concentration of about 1 ppm to about 20 ppm or is added to seafood processing water in an amount sufficient to provide a copper concentration of about 1 ppm to about 20 ppm.

20. The method of claim 19 wherein the disinfection composition has a copper concentration of about 3 ppm or is added to seafood processing water in an amount sufficient to provide a copper concentration of about 3 ppm.

21. The method of claim 1 wherein the disinfection composition comprises (i) sulfuric acid, (ii) ammonium sulfate or sodium sulfate, and (iii) copper sulfate.

22. The method of claim 1 wherein the disinfection composition further comprises a stabilizing agent, wetting agent, hydrotrope, thickener, foaming agent, acidifier, pigment, dye, surfactant, or a combination thereof.

23. The method of claims 1 wherein the disinfection composition consists essentially of ingredients generally recognized as safe (GRAS) food additives.

24. A method of reducing a microbial population on seafood processing equipment comprising the steps of contacting a device used in seafood processing with a disinfection composition comprising an acid, a buffer, and an antimicrobial metal in an amount and time sufficient to reduce a microbial population.

25. A seafood processing system comprising:
a holding station;
a chilling station; and
a sanitization station;
wherein each station is intermittently fluidly connected via a disinfection composition comprising an acid, a buffer, and an antimicrobial metal.
